(19) United States
(12) Patent Application Publication
(10) Pub. No.: US 2015/0313633 A1
(43) Pub. Date: Nov. 5, 2015

(54) PERICARDIAL ACCESS DEVICE

(71) Applicant: RAINBOW MEDICAL LTD., Herzliya (IL)

(72) Inventors: Yossi GROSS, Moshav Mazor (IL); Michael KARDOSH, Kiriat Ono (IL); Amir KISHON, Petach Tikva (IL); Ido SADAN, Tel Aviv (IL); Jonathan YALOM, Tel Aviv (IL); Zev SOHN, Ginot Shomron (IL); Omri GINO, Herzliya (IL)

(21) Appl. No.: 14/324,457

(22) Filed: Jul. 7, 2014

Related U.S. Application Data
(60) Provisional application No. 61/988,457, filed on May 5, 2014.

(51) Int. Cl. A61B 17/34

(52) U.S. Cl. CPC A61B 17/3423; A61B 17/3403; A61B 2017/00247

(57) ABSTRACT

Apparatus and methods are described, including apparatus comprising a longitudinal guide member comprising a blunt distal end having an outer surface at least part of which is transparent. The guide member is advanced distally toward a heart of a subject. A sheath is shaped and sized to surround the guide member and shaped to define an at least partially distally-facing suction port at a distal end of the sheath. A portion of a pericardium of the heart is drawn through the suction port and into the sheath, when the suction port is distal to the guide member. A puncturing element punctures the portion of the pericardium while the portion of the pericardium is in the sheath, and a puncturing-element-restraining element inhibits passage of a distal tip of the puncturing element out of the distal end of the sheath. Other applications are also described.

### CROSS-REFERENCE TO RELATED APPLICATIONS

The present application (a) claims the priority of U.S. Provisional Application No. 61/988,457, entitled “Pericardial Access Device,” filed May 5, 2014, and (b) is related to a provisional application entitled “Left Atrial Appendage Closure” to Gross, filed on even date herewith. Each of the above applications is incorporated herein by reference.

### FIELD OF THE INVENTION

Applications of the present invention relate generally to cardiac procedures and specifically to apparatus and methods for accessing a pericardial region, e.g., a pericardial cavity of a subject.

### BACKGROUND

The heart is enclosed in a double layered membrane termed the pericardium. The pericardium and its serous fluid protect the heart and lubricate the moving surfaces of the heart. The pericardium is composed of two layers: the outermost fibrous pericardium and the inner serous pericardium. The serous pericardium is divided into two layers, the parietal pericardium, which is fused to the fibrous pericardium, and the visceral pericardium (also termed epicardium). Pericardial serous fluid is found in the pericardial cavity (also termed the pericardial space) between the parietal pericardium and visceral layer.

Accessing of the pericardium may facilitate, for example, drug delivery, a pericardiocentesis procedure (aspiration of pericardial fluid typically for diagnosis of a pericardial disease), left atrial appendage removal, coronary artery bypass grafting, or placement of a reflection-facilitation element as described in US 2013/0103028 to Tsoref et al., which is incorporated herein by reference.

Complications may arise during surgical procedures to access the pericardium, and injury may be caused to internal organs such as the liver, stomach and lungs. Therefore, safe and efficient means of accessing the pericardium are desirable.

### SUMMARY OF THE INVENTION

In accordance with some applications of the present invention, apparatus is provided for safely accessing a pericardium of a subject and penetrating the pericardium to access a pericardial region. “Pericardial region,” as used in the present application, including the claims, consists of one or more regions selected from the group consisting of: a region between the pericardium and the myocardium, a region between the fibrous pericardium and the serous pericardium, a region of the pericardial cavity that is between the parietal pericardium and the visceral pericardium (also known as the epicardium).

Accessing of the pericardial region using any of the techniques described herein is useful during procedures such as a pericardiocentesis procedure in which pericardial fluid is aspirated for the purpose of diagnosing a pericardial disease, or for treatment of cardiac tamponade.

Accessing of the pericardial region using any of the techniques described herein may additionally be useful to apply pressure to bleeding myocardial tissue, typically by accessing the pericardial region and applying pressure to the site of bleeding (e.g., by placing a balloon in the pericardial region using the techniques described herein, and inflating the balloon).

The apparatus, as provided by some applications of the present invention, is shaped such as to allow a physician (e.g., an electrophysiologist) to reach the pericardium while avoiding damage to internal organs, including but not limited to, the liver, the diaphragm, the stomach and the lungs. Once the apparatus is in the vicinity of the pericardium, the apparatus contacts an outer surface of the pericardium and applies suction to the pericardium in order to draw a portion of the pericardium into the apparatus. Drawing of the portion of pericardium into the apparatus generally allows for puncturing of the pericardium by the apparatus and accessing of a pericardial region.

The apparatus comprises a longitudinal guide member, e.g., a guide tube, which is advanced distally towards a heart of the subject. The guide tube has a proximal end, a distal end and a guide-tube lumen between the proximal and distal ends. In the context of the present specification and in the claims, “proximal” means closer to the opening through which the guide is inserted into the body, and “distal” means further from this opening. The distal end of the guide tube is shaped as a blunt, typically but not necessarily dome-shaped, distal end. The blunt distal end facilitates advancement of the apparatus towards the heart by separation of tissue by blunt dissection, thereby reducing damage to internal organs. Additionally, at least part of the distal end is transparent, thus facilitating imaging of the procedure by an imaging device disposed, for example, within the guide-tube lumen.

The apparatus further comprises a sheath which is shaped and sized to surround the guide tube and shaped to define an at least partially distally-facing suction port. When the apparatus reaches the vicinity of the heart, the sheath is brought into contact with a portion of the outer surface of the pericardium. Suction is then applied, e.g., via a suction tube in fluid communication with the inside of the sheath, to draw the portion of the pericardium into the suction port of the sheath.

The suction tube typically extends through the guide-tube lumen, and is in contact with a perimeter of a first hole in the distal end of the guide tube. The suction tube draws the portion of the pericardium into the distally-facing suction port of the sheath, by applying suction through the first hole.

The apparatus additionally comprises a needle tube which extends through the guide-tube lumen, and is in contact with a perimeter of a second hole in the distal end of the guide tube. A needle is passed through the needle tube and out of a distal end of the needle tube, in order to puncture the pericardium while the pericardium is in the sheath, gaining access to a pericardial region.

The apparatus further comprises a needle-restraining element which inhibits passage of a distal tip of the needle out of a distal end of the sheath so as to inhibit damage to cardiac tissue by the needle.

There is therefore provided, in accordance with some applications of the present invention, apparatus including:

a longitudinal guide member (a) including a blunt distal end having an outer surface at least part of which is transparent, and (b) configured to be advanced distally toward a heart of a subject;

a sheath shaped and sized to surround the guide member and shaped to define an at least partially distally-facing suction port at a distal end of the sheath, the apparatus being configured to facilitate drawing a portion of a pericardium of the heart through the suction port and into the sheath, when the suction port is distal to the guide member;

a puncturing element configured to puncture the portion of the pericardium while the portion of the pericardium is in the sheath; and

a puncturing-element-restraining element shaped and positioned with respect to the puncturing element to inhibit passage of a distal tip of the puncturing element out of the distal end of the sheath.

In some applications, at least part of the outer surface the distal end of the guide member is dome-shaped.

In some applications,

the guide member is a guide tube having a proximal end,

the distal end is a distal end of the guide tube, and

and the guide tube is shaped to define a guide-tube lumen between the proximal and distal ends of the guide tube.

In some applications, the apparatus further includes an imaging sensor disposed within the guide-tube lumen.

In some applications, the guide-tube lumen has a diameter of 4-15 mm.

In some applications, a radius of curvature at a distalmost point of the distal end of the guide tube is less than a radius of the guide tube.

In some applications, the radius of curvature at the distalmost point of the distal end of the guide tube is 30-60% of the radius of the guide tube.

In some applications, the guide member is a shaft shaped to define one or more longitudinal channels therealong.

In some applications, the apparatus further includes a puncturing-element tube, the puncturing element being sized and shaped to be passable through the puncturing-element tube and out of a distal end of the puncturing-element tube.

In some applications, the distal end of the puncturing-element tube defines a face that is not perpendicular to a local central longitudinal axis of the puncturing-element tube.

In some applications,

the distal end is shaped to define a puncturing-element-tube hole therein, and

the puncturing-element tube is disposed such that the puncturing-element tube is in contact with a perimeter of the puncturing-element-tube hole.

In some applications, the puncturing-element-tube hole has a diameter of 0.2-2 mm.

In some applications, the puncturing-element tube hole has a diameter of 2-5 mm.

In some applications, the puncturing-element tube is disposed between the guide member and the sheath.

In some applications, the puncturing-element tube is a puncturing-element-and-suction tube configured to facilitate the drawing of the portion of the pericardium through the suction port of the sheath by application of suction through the puncturing-element-and-suction tube.

In some applications, the puncturing element is disposed between the guide member and the sheath.

In some applications, the apparatus further includes a suction tube configured to facilitate the drawing of the portion of the pericardium through the suction port of the sheath by application of suction through the suction tube.

In some applications, a distal end of the suction tube defines a face that is not perpendicular to a local central longitudinal axis of the suction tube.

In some applications, the suction tube is disposed between the guide member and the sheath.

In some applications,

the distal end is shaped to define a suction-tube hole therein, and

the suction tube is disposed such that a distal end of the suction tube is in contact with a perimeter of the suction-tube hole.

In some applications, the suction-tube hole has a diameter of 0.2-2 mm.

In some applications, the suction-tube hole has a diameter of 2-5 mm.

In some applications, the apparatus further includes a sensor configured to measure an electrophysiological signal occurring at a tip of the puncturing element.

In some applications, the puncturing element includes a radiofrequency wire, and the apparatus further includes a radiofrequency generator configured to transmit a radiofrequency signal to a distal end of the radiofrequency wire.

In some applications, a diameter of the sheath is between 0.1 and 4 mm greater than a diameter of the guide member.

In some applications, the diameter of the sheath is between 0.2 and 0.6 mm greater than the diameter of the guide member.

In some applications, a 1 cm line extending from a center of the suction port into the sheath, in a direction parallel to a local central longitudinal axis of the sheath, does not contact any part of the apparatus, when the suction port is distal to the guide member.

In some applications, a 1.5 cm line extending from the center of the suction port into the sheath, in the direction parallel to the local central longitudinal axis of the sheath, does not contact any part of the apparatus, when the suction port is distal to the guide member.

In some applications, a 4 mm line extending from a center of the suction port into the sheath, in a direction parallel to a local central longitudinal axis of the sheath, does not contact any part of the apparatus, when the suction port is distal to the guide member.

In some applications, the puncturing element includes a needle shaped to define a lumen thereof.

In some applications, the needle includes a radiofrequency needle, and the apparatus further includes a radiofrequency generator configured to transmit a radiofrequency signal to a distal end of the radiofrequency needle.

In some applications, the apparatus further includes a guidewire shaped to be passable through the lumen of the needle and through a distal end of the needle.

In some applications, the apparatus further includes a needle casing which surrounds the needle, and a distal tip of the needle:

is straight when the needle is surrounded by the casing, and

is curved when the needle is not surrounded by the casing.

In some applications, the apparatus further includes a rigid core structure disposed within the lumen of the needle, and a distal tip of the needle;

is straight when the rigid core structure is disposed within the lumen of the needle, and

is curved when the rigid core structure is not disposed within the lumen of the needle.

In some applications, the apparatus further includes an imaging device disposed at least partially at the distal end of the guide member.

In some applications, the imaging device includes an imaging sensor disposed at the distal end of the guide member.

In some applications, the imaging device includes:

a fiber optic array having a distal end that is disposed at the distal end of the guide member; and

an imaging sensor coupled to a proximal end of the fiber optic array.

In some applications, at least part of the imaging device is disposed within 15 mm of a distalmost point of the distal end of the guide member.

In some applications, the apparatus further includes at least one illumination-providing element disposed at least partially at the distal end of the guide member and configured to provide illumination for operation of the imaging device.

In some applications, the at least one illumination-providing element includes a light source disposed at the distal end of the guide member.

In some applications, the at least one illumination-providing element includes:

an optical fiber having a distal end that is disposed at the distal end of the guide member; and

a light source coupled to a proximal end of the optical fiber.

In some applications, at least 80% of light that is emitted from the at least one illumination-providing element and reflected by the distal end of the guide member is not directly reflected to the imaging device, due to a factor selected from the group consisting of: a disposition of the at least one illumination-providing element with respect to the imaging device, a shape of the distal end of the guide member, an optical parameter of the distal end of the guide member, and an optical parameter of a coating on the distal end of the guide member.

In some applications, the apparatus further includes one or more polarizing filters at least partially covering an element of the apparatus selected from the group consisting of: the at least one illumination-providing element, the imaging device, and an inner surface of the distal end.

In some applications, the apparatus further includes a heart-proximity sensor configured to generate a signal indicative of a proximity of the distal end of the guide member to the heart of the subject.

In some applications, the heart-proximity sensor includes an ultrasound sensor.

In some applications, the heart-proximity sensor includes a contact sensor configured to generate a signal indicative of contact of the distal end of the guide member to the heart of the subject.

In some applications, the heart-proximity sensor includes an accelerometer.

In some applications, the apparatus further includes a processor configured to (a) detect a component of the signal having a frequency between 0.5 and 3 Hz and a magnitude that is greater than a threshold, and (b) in response to the detecting, generate an output indicative of the proximity of the distal end of the guide member to the heart of the subject.

In some applications, the processor is configured to generate the output only if the detected component of the signal corresponds to a direction that is generally perpendicular to a plane defined by the suction port of the sheath.

In some applications, the processor is configured to generate the output only if the detected component of the signal corresponds to a direction that is generally parallel to a central longitudinal axis of the guide member at the distal end of the guide member.

In some applications, the heart-proximity sensor further includes a contact sensor, and the heart-proximity sensor is further configured to generate a signal indicative of a proximity of the distal end of the guide member to an internal organ that is not the heart of the subject, by (a) the contact sensor generating a signal that is indicative of contact of the contact sensor with the internal organ, and (b) the accelerometer not generating a signal having a frequency between 0.5 and 3 Hz and a magnitude that is greater than a threshold.

In some applications, an angle between (a) a local central longitudinal axis of the sheath, and (b) a normal to a plane defined by the suction port of the sheath, is between 40 and 70 degrees.

In some applications, an angle between (a) a local central longitudinal axis of the sheath, and (b) a normal to a plane defined by the suction port of the sheath, is between 0 and 50 degrees.

In some applications, the angle is between 10 and 40 degrees.

In some applications, the distal end of the guide member is rotationally asymmetric with respect to a local central longitudinal axis of the guide member.

In some applications, at at least one site within 1 mm of a distalmost point of the distal end of the guide member, a centroid of a cross-section of the distal end of the guide member does not lie on the local central longitudinal axis of the guide member.

In some applications, at all sites within 3 mm of a distalmost point of the distal end of the guide member, a centroid of a cross-section of the distal end of the guide member does not lie on the local central longitudinal axis of the guide member.

In some applications, a radius of curvature at a distalmost point of the distal end of the guide member is between 0.5 and 5 mm.

In some applications, the apparatus further includes an o-ring disposed at the suction port of the sheath.

In some applications, the apparatus further includes a vibrating element configured to vibrate the distal end of the guide member.

There is further provided, in accordance with some applications of the present invention, a method including:

distally advancing a guide member toward a heart of a subject, the guide member having a distal end thereof;

deploying a sheath over the guide member, the sheath being shaped to define a suction port at a distal end thereof;

contacting an outer surface of a pericardium of the heart with a perimeter of the suction port;

drawing a portion of the pericardium into the sheath by applying suction to the pericardium through the suction port; and

using a puncturing element, puncturing the portion of the pericardium that is in the sheath.

In some applications, applying suction to the pericardium includes applying suction by applying an oscillating suction pressure.

In some applications, applying suction to the pericardium includes applying a suction pressure that increases at an average rate of between 5 and 15 mm Hg per second, for at least 1 second.

In some applications, the method further includes, before deploying the sheath over the guide member;

using a position sensor disposed at a distal portion of the guide member to measure a position of the distal end of the guide member; and

ascertaining a proximity of the distal end of the guide member to the heart of the subject, using the measured position.

In some applications, distally advancing the guide member toward the heart of the subject includes:

using a position sensor disposed at a distal portion of the guide member to measure a position of the distal end of the guide member; and

using the measured position to navigate the guide member.

In some applications, distally advancing the guide member toward the heart of the subject includes:

using a position-and-orientation sensor disposed at a distal portion of the guide member to measure a position and an orientation of the distal end of the guide member; and

using the measured position and orientation to navigate the guide member.

In some applications, distally advancing the guide member toward the heart of the subject includes:

using an ultrasound sensor disposed at a distal portion of the guide member to generate an image; and

using the image to navigate the guide member.

In some applications, distally advancing the guide member toward the heart of the subject includes using a preoperative image of the subject to navigate the guide member.

In some applications, the method further includes, following the puncturing:

using a sensor to measure an electrophysiological signal occurring at a tip of the puncturing element, and

ascertaining that the puncturing element has punctured the portion of the pericardium, in response to the measuring.

In some applications, applying suction to the pericardium includes applying suction through a space that is between an outer wall of the guide member and an inner wall of the sheath.

In some applications, applying suction to the pericardium includes applying suction through a suction tube.

In some applications, the puncturing element includes a radiofrequency wire, and using the puncturing element to puncture the portion of the pericardium includes using a radiofrequency generator to transmit a radiofrequency signal to a distal end of the radiofrequency wire.

In some applications, the method further includes using a puncturing-element-restraining element to inhibit passage of a distal tip of the puncturing element out of the distal end of the sheath.

In some applications, the puncturing element includes a needle, and using the puncturing element includes using the needle to puncture the portion of the pericardium.

In some applications, the needle includes a radiofrequency needle, and using the puncturing element to puncture the portion of the pericardium includes using a radiofrequency generator to transmit a radiofrequency signal to a distal end of the radiofrequency needle.

In some applications, the method further includes passing a guidewire through a lumen of the needle, following the puncturing of the portion of the pericardium.

In some applications, the method further includes, following the passing of the guidewire through the lumen of the needle:

withdrawing the needle;

passing a tube over the guidewire;

withdrawing the guidewire; and

passing a tool through the tube.

In some applications, the method further includes, following the passing of the guide wire through the lumen of the needle:

withdrawing the needle; and

passing a tool over the guidewire.

In some applications, the tool includes a reflection-facilitation element, and the method further includes, following the passing of the tool over the guidewire:

placing the reflection-facilitation element between a myocardium of the heart and the pericardium; and

transmitting an ultrasound signal from within a chamber of the heart, toward the reflection-facilitation element.

In some applications, the tool includes an expandable element, and passing the tool over the guidewire includes passing the expandable element over the guidewire.

In some applications, the method further includes, following the passing of the expandable element over the guidewire, expanding the expandable element between a myocardium of the heart and the pericardium.

In some applications, the expandable element includes an inflatable element, and expanding the expandable element includes inflating the inflatable element.

In some applications, the expandable element includes an expandable mesh, and expanding the expandable element includes expanding the expandable mesh.

In some applications, the method further includes, following the expanding of the expandable element, inhibiting bleeding of the heart by applying pressure with the expandable element.

In some applications, expanding the expandable element includes creating a working space between a myocardium of the heart and the pericardium, and the method further includes passing a surgical tool over the guidewire and into the working space.

In some applications, the method further includes, before deploying the sheath over the guide member:

using an imaging device disposed at least partially at the distal end of the guide member to generate an image of at least part of the heart of the subject; and

ascertaining a proximity of the distal end of the guide member to the heart of the subject, using the image.

In some applications, the method further includes rinsing a lens of the imaging device by passing rinsing fluid through a rinse port that is adjacent to the lens.

In some applications, the method further includes using at least one illumination-providing element disposed at least partially at the distal portion of the guide member to provide illumination for operation of the imaging device.

In some applications, using the at least one illumination-providing element to provide illumination for the operation of the imaging device includes:

ascertaining a level of glare in the image; and

in response to the level of glare, moving the illumination-providing element.

In some applications, the at least one illumination-providing element includes a first illumination-providing element and a second illumination-providing element, and using the at least one illumination-providing element to provide illumination for the operation of the imaging device includes:

ascertaining a level of glare in the image;

in response to the level of glare, selecting exactly one illumination-providing element from the first illumination-providing element and the second illumination-providing element; and

providing illumination from the selected illumination-providing element.

In some applications, the method further includes, before deploying the port of the sheath over the guide member:

using a heart-proximity sensor to generate a signal; and

ascertaining a proximity of the distal end of the guide member to the heart of the subject, in response to the signal.

In some applications, the heart-proximity sensor includes an ultrasound sensor, and using the heart-proximity sensor to generate the signal includes using the ultrasound sensor to generate the signal.

In some applications, the heart-proximity sensor includes a contact sensor, and using the heart-proximity sensor to generate the signal includes using the contact sensor to generate a signal indicative of contact of the distal end of the guide member with the heart of the subject.

In some applications,

the distal end of the guide member is covered by a cover,

the heart-proximity sensor includes a contact sensor, and

using the heart-proximity sensor to generate the signal includes using the contact sensor to generate a signal indicative of contact of the cover with the heart of the subject.

In some applications, the heart-proximity sensor includes an accelerometer, and using the heart-proximity sensor to generate the signal includes using the accelerometer to generate the signal.

In some applications, the method further includes using a processor to (a) detect a component of the signal having a frequency between 0.5 and 3 Hz and a magnitude that is greater than a threshold, and (b) in response to the detecting, generate an output indicative of the proximity of the distal end of the guide member to the heart of the subject.

In some applications, using the processor to generate the output includes using the processor to generate the output in response to the detected component of the signal corresponding to a direction that is generally perpendicular to a plane defined by the suction port of the sheath.

In some applications, using the processor to generate the output includes using the processor to generate the output in response to the detected component of the signal corresponding to a direction that is generally parallel to a central longitudinal axis of the guide member at the distal end of the guide member.

In some applications, the heart-proximity sensor further includes a contact sensor, and the method further includes, upon the distal end of the guide member being proximate to an internal organ that is not the heart of the subject, using the heart-proximity sensor to generate a signal indicative of the proximity of the distal end of the guide member to the internal organ, by (a) the contact sensor generating a signal that is indicative of contact of the contact sensor with the internal organ, and (b) the accelerometer not generating a signal having a frequency between 0.5 and 3 Hz and a magnitude that is greater than a threshold.

In some applications,

the distal end of the guide member is covered by a cover,

the heart-proximity sensor further includes a contact sensor, and

the method further includes, upon the cover being proximate to an internal organ that is not the heart of the subject using the heart-proximity sensor to generate a signal indicative of the proximity of the cover to the internal organ, by (a) the contact sensor generating a signal that is indicative of contact of the contact sensor with the internal organ, and (b) the accelerometer not generating a signal having a frequency between 0.5 and 3 Hz and a magnitude that is greater than a threshold.

There is further provided, in accordance with some applications of the present invention, apparatus including:

a longitudinal guide member configured to be advanced distally toward a heart of a subject, the guide member having a distal end thereof;

a distal-end cover shaped to (i) define an aperture therein, and (ii) cover the distal end of the guide member;

a sheath shaped and sized to surround the guide member and shaped to define an at least partially distally-facing suction port at a distal end of the sheath, the apparatus being configured to facilitate drawing a portion of a pericardium of the heart through the suction port and into the sheath;

a puncturing element configured to puncture the portion of the pericardium while the portion of the pericardium is in the sheath;

an imaging device disposed at the distal end of the guide member and facing the aperture; and

a puncturing-element-restraining element shaped and positioned with respect to the puncturing element to inhibit passage of a distal tip of the puncturing element out of the distal end of the sheath.

In some applications, the distal end of the guide member is shaped to define a rinse port therein, and the apparatus further includes a rinse tube in fluid communication with the rinse port.

In some applications, the rinse tube is a rinse-and-suction tube configured to facilitate the drawing of the portion of the pericardium through the suction port of the sheath by application of suction through the rinse-and-suction tube.

In some applications, the apparatus further includes a puncturing-element tube, the puncturing element being sized and shaped to be passable through the puncturing-element tube and out of a distal end of the puncturing-element tube.

In some applications, the distal end of the puncturing-element tube defines a face that is not perpendicular to a local central longitudinal axis of the puncturing-element tube.

In some applications, the puncturing-element tube is disposed between the guide member and the sheath.

In some applications, the puncturing-element tube is a puncturing-element-and-suction tube configured to facilitate the drawing of the portion of the pericardium through the suction port of the sheath by application of suction through the puncturing-element-and-suction tube.

In some applications,

the guide member is a guide tube having a proximal end,

the distal end is a distal end of the guide tube, and

the guide tube is shaped to define a guide-tube lumen between the proximal and distal ends of the guide tube.

In some applications, the guide-tube lumen has a diameter of 4-15 mm.

In some applications, the apparatus further includes a suction tube configured to facilitate the drawing of the portion of the pericardium through the suction port of the sheath by application of suction through the suction tube.

In some applications, a distal end of the suction tube defines a face that is not perpendicular to a local central longitudinal axis of the suction tube.

In some applications, the suction tube is disposed between the guide member and the sheath.

In some applications, the apparatus further includes a sensor configured to measure an electrophysiological signal occurring at a tip of the puncturing element.

In some applications, the puncturing element includes a radiofrequency wire, and the apparatus further includes a radio-frequency generator configured to transmit a radiofrequency signal to a distal end of the radiofrequency wire.

In some applications, a diameter of the sheath is between 0.1 and 4 mm greater than a diameter of the guide member.

In some applications, the diameter of the sheath is between 0.2 and 0.6 mm greater than the diameter of the guide member.

In some applications, a 1 cm line extending from a center of the suction port into the sheath, in a direction parallel to a local central longitudinal axis of the sheath, does not contact any part of the apparatus, when the suction port is distal to the guide member.

In some applications, a 1.5 cm line extending from the center of the suction port into the sheath, in the direction parallel to the local central longitudinal axis of the sheath, does not contact any part of the apparatus, when the suction port is distal to the guide member.

In some applications, a 4 mm line extending from a center of the suction port into the sheath, in a direction parallel to a local central longitudinal axis of the sheath, does not contact any part of the apparatus, when the suction port is distal to the guide member.

In some applications, the puncturing element includes a needle shaped to define a lumen thereof.

In some applications, the needle includes a radiofrequency needle, and the apparatus further includes a radiofrequency generator configured to transmit a radiofrequency signal to a distal end of the radiofrequency needle.

In some applications, the apparatus further includes a guidewire shaped to be passable through the lumen of the needle and through a distal end of the needle.

In some applications, the apparatus further includes a needle casing which surrounds the needle, and a distal tip of the needle:

is straight when the needle is surrounded by the casing, and

is curved when the needle is not surrounded by the casing.

In some applications, the apparatus further includes a rigid core structure disposed within the lumen of the needle, and a distal tip of the needle:

is straight when the rigid core structure is disposed within the lumen of the needle, and

is curved when the rigid core structure is not disposed within the lumen of the needle.

In some applications, the apparatus further includes at least one illumination-providing element disposed at least partially at the distal end of the guide member and configured to provide illumination for operation of the imaging device.

In some applications, the apparatus further includes a heart-proximity sensor configured to generate a signal indicative of a proximity of the distal-end cover to the heart of the subject.

In some applications, the heart-proximity sensor includes an ultrasound sensor.

In some applications, the heart-proximity sensor includes a contact sensor configured to generate a signal indicative of contact of the distal-end cover with the heart of the subject.

In some applications, the heart-proximity sensor includes an accelerometer.

In some applications, the apparatus further includes a processor configured to (a) detect a component of the signal having a frequency between 0.5 and 3 Hz and a magnitude that is greater than a threshold, and (b) in response to the detecting, generate an output indicative of the proximity of the distal-end cover to the heart of the subject.

In some applications, the processor is configured to generate the output only if the detected component of the signal corresponds to a direction that is generally perpendicular to a plane defined by the suction port of the sheath.

In some applications, the processor is configured to generate the output only if the detected component of the signal corresponds to a direction that is generally parallel to a central longitudinal axis of the guide member at the distal end of the guide member.

In some applications, the heart-proximity sensor further includes a contact sensor, and the heart-proximity sensor is further configured to generate a signal indicative of a proximity of the distal-end cover to an internal organ that is not the heart of the subject, by (a) the contact sensor generating a signal that is indicative of contact of the contact sensor with the internal organ, and (b) the accelerometer not generating a signal having a frequency between 0.5 and 3 Hz and a magnitude that is greater than a threshold.

In some applications, an angle between (a) a local central longitudinal axis of the sheath, and (b) a normal to a plane defined by the suction port of the sheath, is between 40 and 70 degrees.

In some applications, an angle between (a) a local central longitudinal axis of the sheath, and (b) a normal to a plane defined by the suction port of the sheath, is between 0 and 50 degrees.

In some applications, the angle is between 10 and 40 degrees.

In some applications, the apparatus further includes an o-ring disposed at the suction port of the sheath.

In some applications, the apparatus further includes a vibrating element configured to vibrate the distal-end cover.

There is further provided, in accordance with some applications of the present invention, apparatus for applying pressure between two layers of tissue, the apparatus including:

a flexible longitudinal element shaped to define a lumen thereof;

an expandable element disposed at a distal portion of the flexible longitudinal element, the expandable element being shaped to define, upon being expanded, a disk, and configured to apply pressure upon being expanded; and

an imaging device disposed at least partially at a distal portion of the apparatus.

There is further provided, in accordance with some applications of the present invention, apparatus for creating a working space between two layers of tissue, the apparatus including:

a flexible longitudinal element shaped to define a lumen thereof;

an expandable element disposed at a distal portion of the flexible longitudinal element the expandable element shaped to define and at least partly surround the working space, upon the expandable element being expanded; and

an imaging device disposed at least partially at a distal portion of the apparatus.

In some applications, the imaging device includes an imaging sensor disposed at the distal portion of the apparatus.

In some applications, the imaging device includes:

a fiber optic array having a distal end that is disposed at the distal portion of the apparatus; and

an imaging sensor coupled to a proximal end of the fiber optic array.

In some applications, the fiber optic array is shaped to be passable through the lumen of the flexible longitudinal element.

In some applications, the imaging device is coupled to an element selected from the group consisting of: the flexible longitudinal element, and the expandable element.

In some applications, the imaging device includes an Imaging sensor that is shaped to be passable through the lumen of the flexible longitudinal element.

In some applications, the apparatus further includes a surgical tool shaped to be passable through the lumen of the flexible longitudinal element and into the working space.

In some applications, the expandable element includes an expandable mesh.

In some applications, the expandable mesh is shaped to define a concave shape upon being expanded.

In some applications, the expandable element includes an inflatable element.

In some applications, the expandable element is shaped to define, upon being expanded, a ring.

In some applications, the expandable element is shaped to define, upon being expanded, a partial ring.

In some applications, the expandable element is sized and shaped to be containable within a rectangle having (a) a length between 3 and 8 cm, and (b) a width between 3 and 8 cm, upon the expandable element being expanded.

In some applications, the expandable element is configured to, upon being expanded, have a greater cross-sectional area at a proximal portion thereof, relative to a distal portion thereof.

In some applications, the expandable element is configured to, upon being expanded, have a greater cross-sectional area at a distal portion thereof, relative to a proximal portion thereof.

In some applications, the expandable element is configured to, upon being expanded, have a greater cross-sectional area at a middle portion thereof, relative to (a) a proximal portion thereof, and (b) a distal portion thereof.

There is further provided, in accordance with some applications of the present invention, a method for performing a procedure in an area between two layers of tissue, the method including:

creating a working space, by expanding an expandable element in the area such that the expandable element defines and at least partly surrounds the working space;

passing a tool into the working space; and

using the tool to perform the procedure.

In some applications, the two layers of tissue include a myocardium of a heart and a pericardium of the heart, and performing the procedure includes performing the procedure in an area between the myocardium and the pericardium.

In some applications, the two layers of tissue include two layers of meninges, and performing the procedure includes performing the procedure in an area between the two layers of meninges.

In some applications, the expandable element is disposed at a distal portion of a flexible longitudinal element, and the method further includes using an imaging device coupled to an element selected from the group consisting of: the flexible longitudinal element, and the expandable element, to image the working space.

In some applications, the method further includes reducing flow of blood in a blood vessel, by applying pressure to the blood vessel with the expandable element.

In some applications, reducing flow of blood in the blood vessel includes reducing flow of blood toward the working space.

In some applications, reducing flow of blood in the blood vessel includes reducing flow of blood away from the working space.

In some applications, applying pressure to the blood vessel with the expandable element includes applying pressure to the blood vessel with a proximal portion of the expandable element.

In some applications, performing the procedure includes at least partially removing a left atrial appendage that is at least partially contained within the working space.

In some applications, performing the procedure includes applying a suture to a blood vessel, and the method further includes testing a resistance of the suture to pressure, by applying pressure to the blood vessel with the expandable element.

In some applications, expanding the expandable element in the area includes expanding an expandable mesh.

In some applications, passing the tool into the working space includes passing the tool through the expandable mesh.

In some applications, expanding the expandable element in the area includes inflating an inflatable element.

There is further provided, in accordance with some applications of the present invention, apparatus including:

a longitudinal guide member configured to be advanced distally toward a heart of a subject, the guide member including a distal end including:
an outer tube-wall shaped to define a lumen thereof; and
an inner tube-wall disposed within the lumen of the outer tube-wall,

the apparatus being configured to facilitate the drawing of a portion of a pericardium of the heart into a portion of the lumen of the outer tube-wall that is between the outer tube-wall and inner tube-wall, by application of suction through the portion of the lumen of the outer tube-wall;

a puncturing element configured to puncture the portion of the pericardium while the portion of the pericardium is in the portion of the lumen of the outer tube-wall; and

a puncturing-element-restraining element shaped and positioned with respect to the puncturing element to inhibit passage of a distal tip of the puncturing element out of the distal end of the guide tube.

In some applications, the distal end further includes a blunt dome-shaped cover at least part of which is transparent, the cover being disposed at a distal end of the inner tube-wall and covering space that is inside the inner tube-wall.

In some applications, an inner diameter of the outer tube-wall is 0.2-4 mm greater than an outer diameter of the inner tube-wall.

In some applications, a distalmost perimeter of the inner tube-wall is 0.2-4 mm distal to a distalmost perimeter of the outer tube-wall.

There is further provided, in accordance with some applications of the present invention, apparatus for use with a catheter, the apparatus including:

a heart-proximity sensor including:
an accelerometer; and
a contact sensor,

the heart-proximity sensor being configured to (a) be disposed at a distal portion of the catheter, and (b) generate a signal indicative of a proximity of the distal portion of the catheter to an internal organ of a subject that is not a heart of the subject, in response to (a) the contact sensor generating a signal that is indicative of contact of the contact sensor with the internal organ, and (b) the accelerometer not generating a signal having a frequency between 0.5 and 3 Hz and a magnitude that is greater than a threshold.

There is further provided, in accordance with some applications of the present invention, apparatus for use with a catheter, the apparatus including:

an accelerometer configured to (a) be disposed at a distal portion of the catheter, and (b) generate a signal indicative of a proximity of the distal portion of the catheter to a heart of a subject; and

a processor configured to (a) detect a component of the signal having a frequency between 0.5 and 3 Hz and a magnitude that is greater than a threshold, and (b) in response to the detecting, generate an output indicative of the proximity of the distal portion of the catheter to the heart of the subject, only if the detected component of the signal corresponds to a direction that is generally parallel to a local central longitudinal axis of the catheter.

The present invention will be more fully understood from the following detailed description of embodiments thereof, taken together with the drawings, in which:

### BRIEF DESCRIPTION OF THE DRAWINGS

FIG. 1 is a schematic illustration of apparatus for penetrating a pericardium, in accordance with some applications of the present invention;

FIGS. 2A-E are schematic illustrations of respective apparatus for penetrating a pericardium, in accordance with some applications of the present invention;

FIGS. 3A-B are schematic illustrations of longitudinal cross-sections of apparatus for penetrating a pericardium, in accordance with some applications of the present invention;

FIG. 3C is a schematic illustration of a sheath and suction port, in accordance with some applications of the present invention;

FIG. 3D is a schematic illustration of a cross-section of apparatus for penetrating a pericardium, in accordance with some applications of the present invention;

FIG. 3E is a schematic illustration of a puncturing-element tube, in accordance with some applications of the present invention;

FIGS. 4A-C are schematic illustrations of longitudinal cross-sections of apparatus for penetrating a pericardium, further showing a puncturing-element-restraining element in accordance with some applications of the present invention;

FIGS. 5A-E show use of apparatus for penetrating a pericardium, in accordance with some applications of the present invention;

FIG. 6 is a schematic illustration of an inflatable element for use with apparatus for penetrating a pericardium, in accordance with some applications of the present invention;

FIGS. 7A-B and 8A-B are schematic illustrations of a curved needle for use with applications of the present invention;

FIG. 9 is a schematic illustration of apparatus that creates a working space between two layers of tissue, in accordance with some applications of the present invention;

FIG. 10 is a schematic illustration of an asymmetric distal end of a longitudinal guide member, in accordance with some applications of the present invention;

FIG. 11 is a schematic illustration of apparatus that creates a working space between two layers of tissue, in accordance with some applications of the present invention;

FIGS. 12A-G show respective designs of an expandable element, in accordance with some applications of the present invention;

FIGS. 13A-B are schematic illustrations of apparatus being used to reduce flow of blood in a blood vessel, in accordance with some applications of the present invention;

FIG. 14A is a schematic illustration of proximity-sensing apparatus, in accordance with some applications of the present invention; and

FIGS. 14B-C are flow charts of proximity-sensing methods, in accordance with some applications of the present invention.

### DETAILED DESCRIPTION OF APPLICATIONS

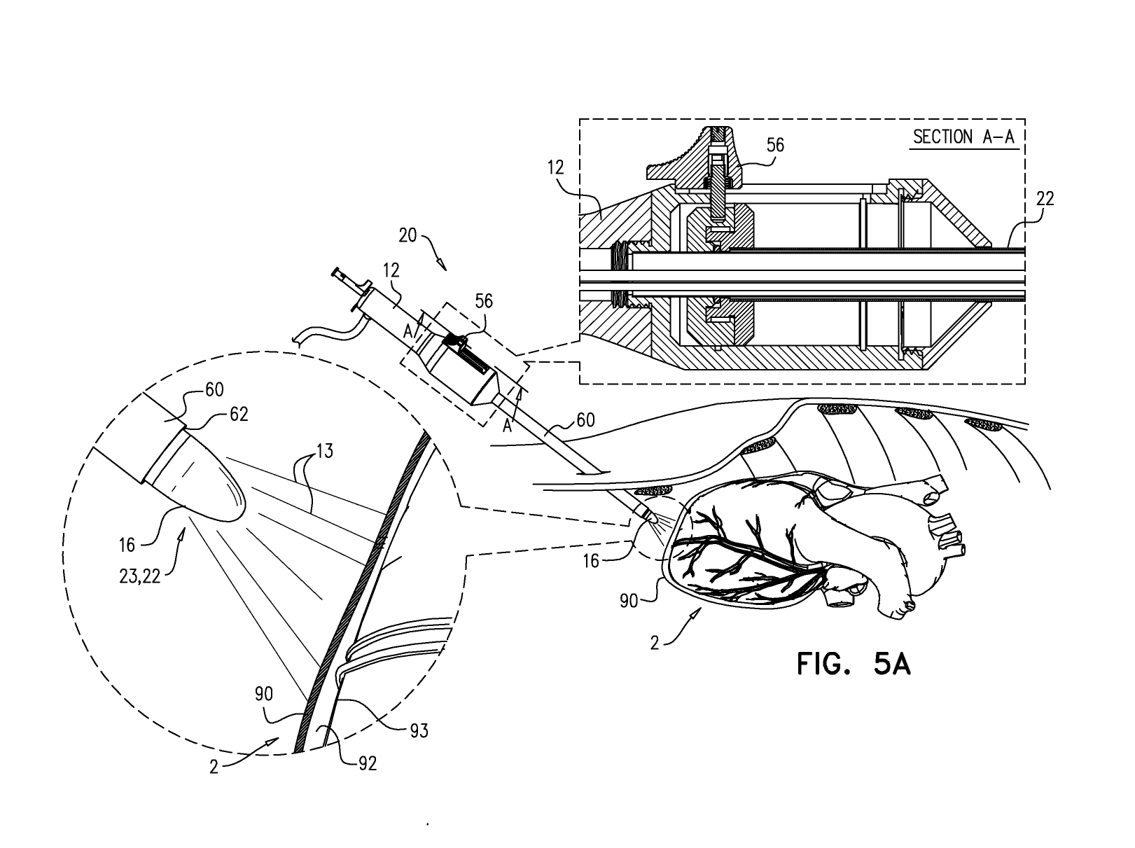

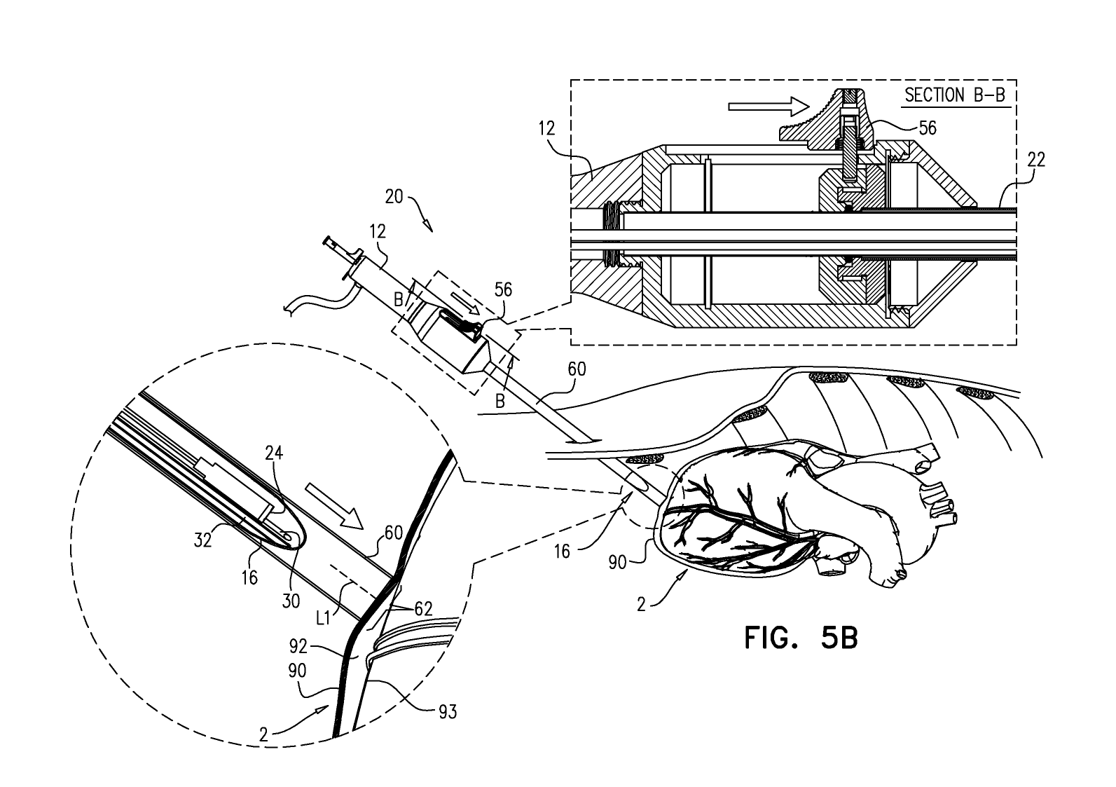

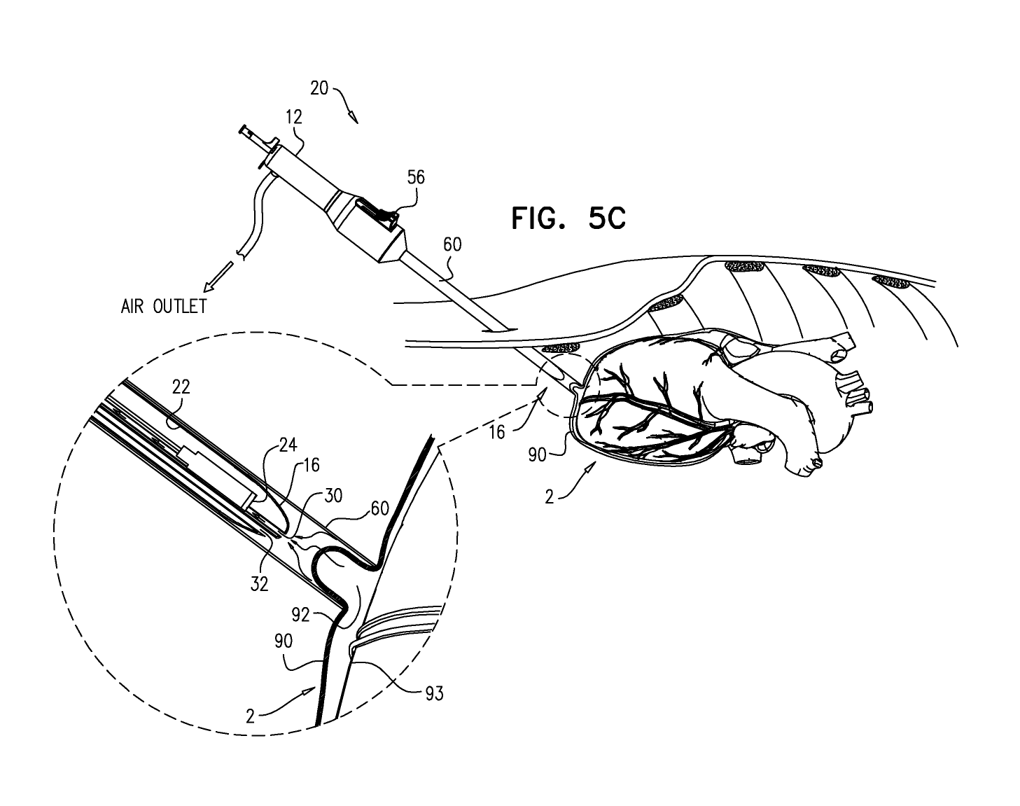

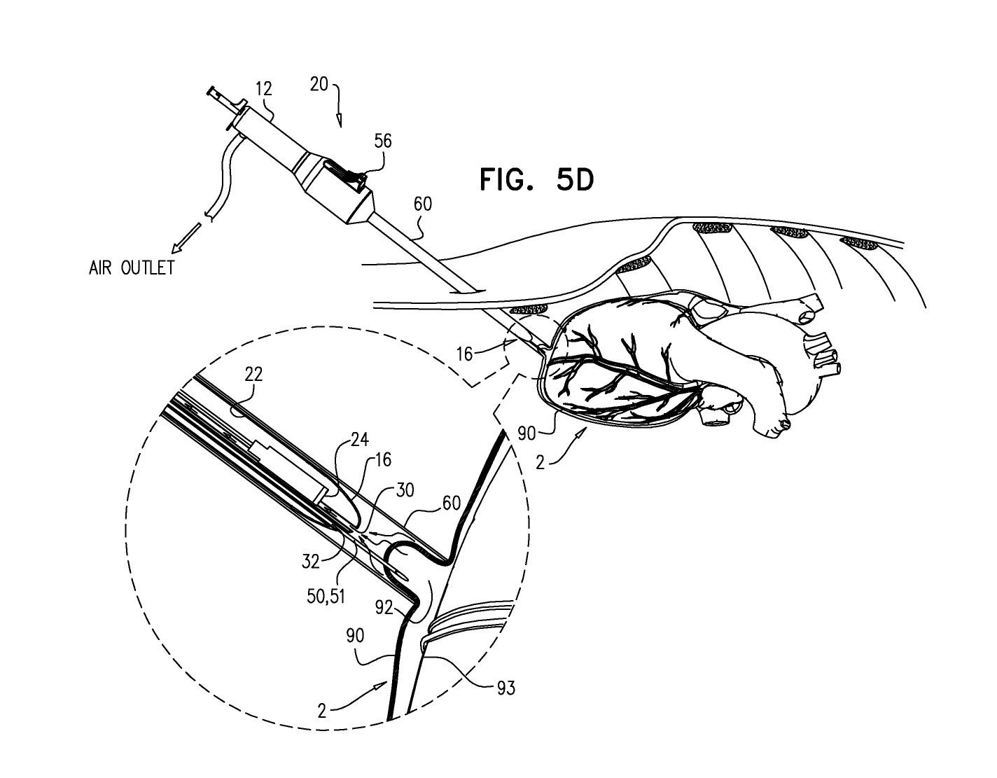

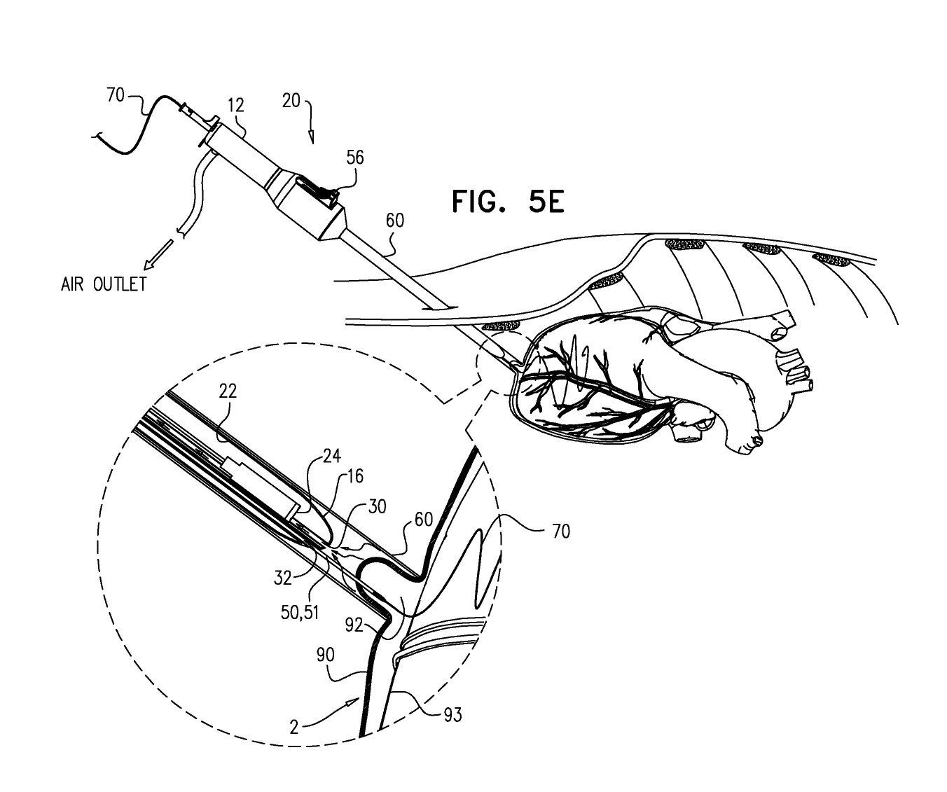

The present description begins with a general overview of a method for accessing a pericardial region, in accordance with some applications of the present invention, as depicted in FIGS. 5A-E.

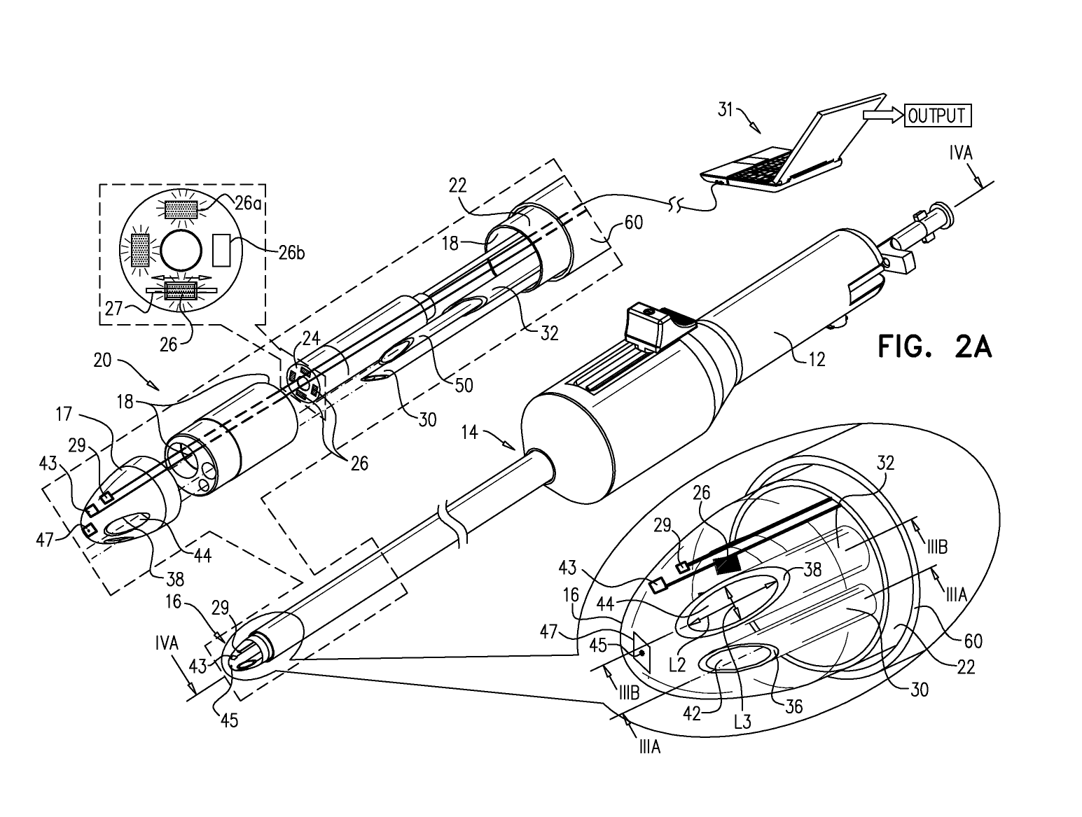

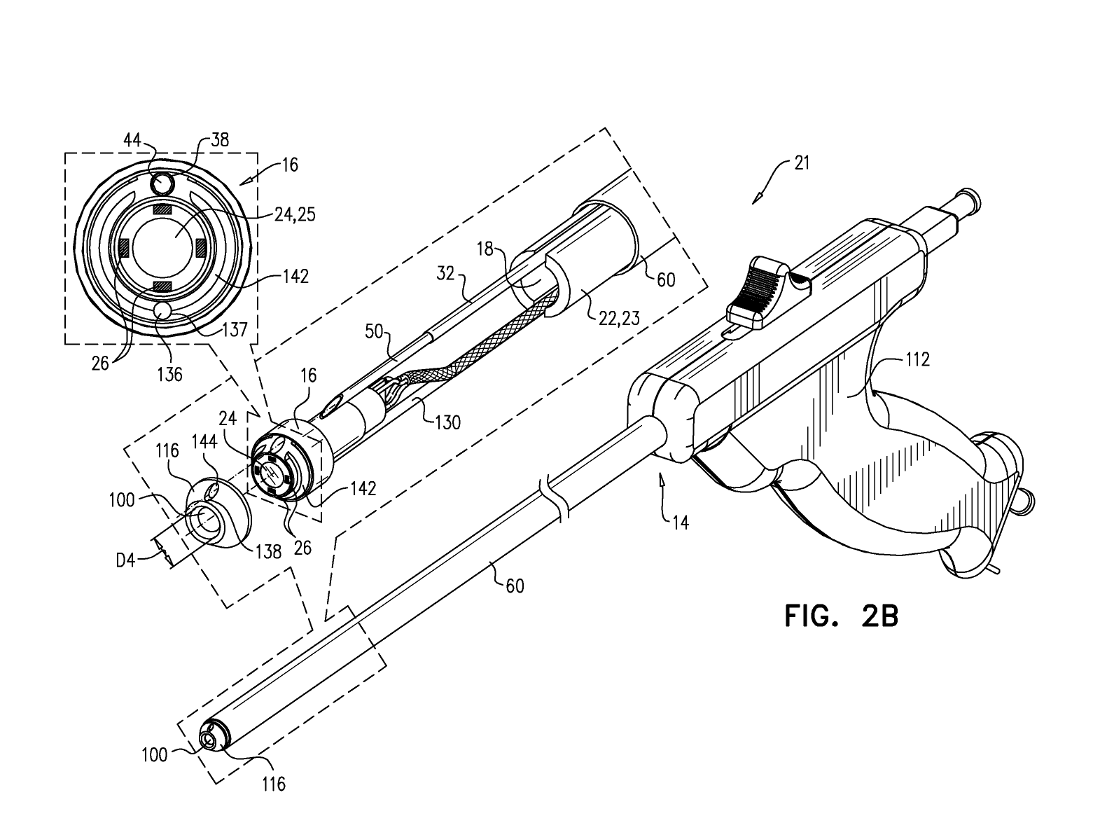

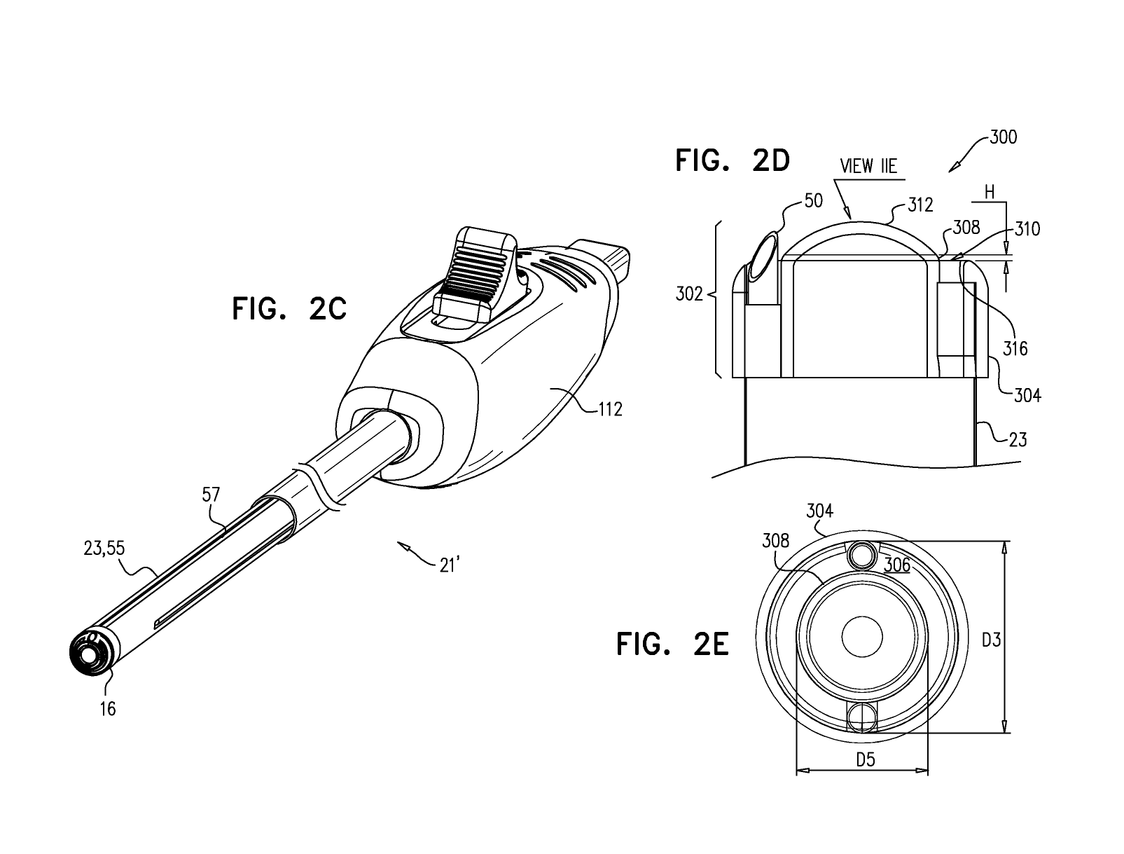

FIGS. 5A-E show use of apparatus 20 for penetrating a pericardium 90 of a subject, and accessing a pericardial region 92. FIG. 5A shows a longitudinal guide member 23, e.g., a guide tube 22, of apparatus 20 being distally advanced toward a heart 2 of the subject, a distal end 16 of guide tube 22 emerging from a distal suction port 62 of sheath 60. Advancement of guide tube 22 may be facilitated by an imaging device 24 (FIG. 5B). Typically, at least one illumination-providing element 26 (FIG. 2A) provides illumination for imaging device 24, the illumination being depicted in FIG. 5A by light rays 13.

When guide tube 22 reaches the heart, the operating physician deploys sheath 60 over the guide tube, e.g., by sliding a slide bar 56 distally, and a perimeter of suction port 62 contacts an outer surface of pericardium 90 (FIG. 5B). Suction is then applied to the pericardium through suction port 62, e.g., via a suction tube 30, and a portion of pericardium 90 is drawn into the sheath, as shown in FIG. 5C. (Suction may be applied using a hospital suction generator, and/or an external vacuum pump, and/or a syringe.) For some applications, pericardial tissue is drawn at least 4 mm into sheath 60, e.g., at least 1 cm or at least 1.5 cm into sheath 60.

After the portion of pericardium 90 is drawn into sheath 60, a puncturing element 50 (e.g., a needle 51) is advanced distally to puncture the portion of the pericardium, as shown in FIG. 5D. The puncturing of the portion of the pericardium provides access to pericardial region 92, e.g., a region between pericardium 90 and myocardial tissue 93. Optionally, grasping elements, such as forceps and/or other types of grasping elements (e.g., corkscrew-like or screw-shaped grasping elements), may be employed to grip the portion of pericardium that is inside the sheath, to facilitate the puncturing.

For some applications, following the puncturing of pericardium 90, a guidewire 70 is advanced through a lumen of needle 51 and into pericardial region 92 (FIG. 5E). Typically, the needle is then withdrawn, and a tool is passed over the guidewire, as further described hereinbelow. Alternatively or additionally, needle 51 delivers a fluid, e.g., a gas for inflation of the pericardium, and/or a contrast medium, to the pericardial region.

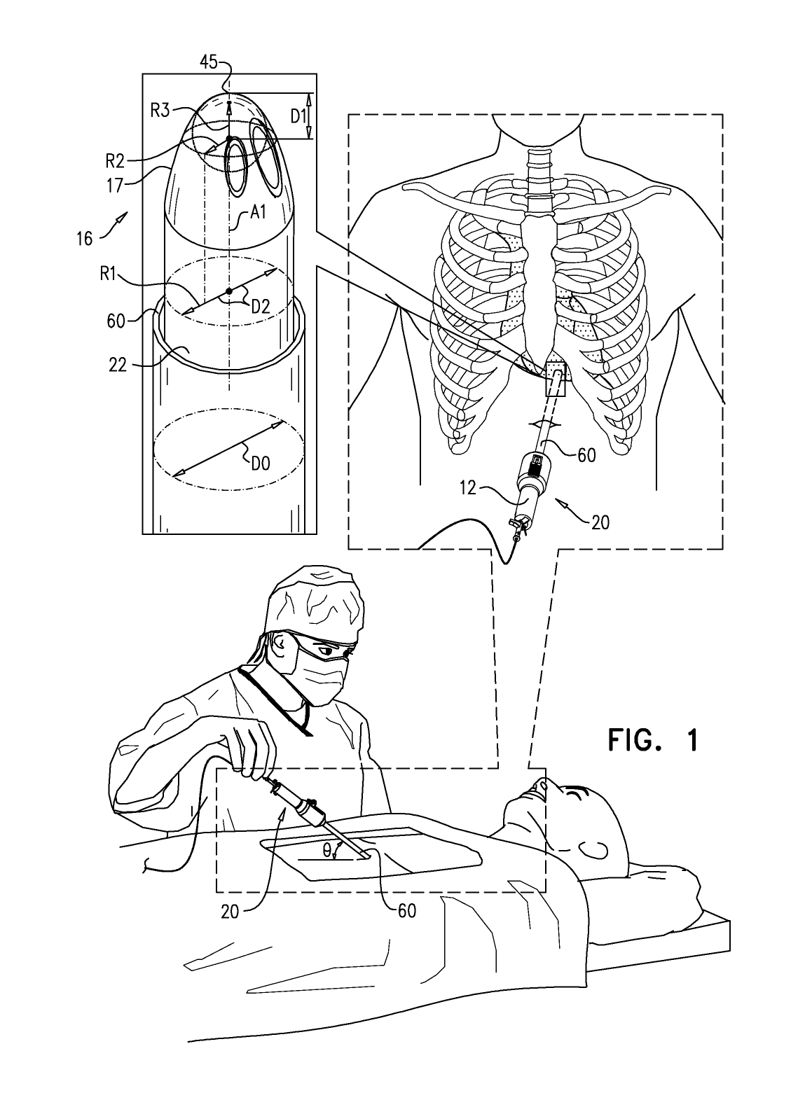

Reference is now made to FIG. 1, which is a schematic illustration of apparatus 20, as provided in accordance with some application of the present invention. Apparatus 20 is inserted into the subject, and is advanced distally toward the heart of the subject. It is noted that apparatus 20 may be advanced towards the heart through any suitable pathway. For example, apparatus 20 may be advanced through the subxiphoid incision, above the diaphragm, directly to the heart.

Apparatus 20 is generally shaped to provide safe and efficient access to the heart. For example, distal end 16 of guide tube 22, as shown in FIG. 1, is typically blunt, the bluntness of the distal end generally facilitating blunt dissection of tissue (i.e., generally atraumatic separation of adjacent tissues) during advancement toward the heart, and generally reducing the chances of injury to internal organs such as the diaphragm, the lungs, the stomach and the liver. Typically, blunt distal end 16 is sufficient in size to allow blunt dissection. In some applications, as shown in FIG. 1, at least part of an outer surface 17 of distal end 16 is dome-shaped. A radius of curvature R3 at the distalmost point 45 of distal end 16 is typically at least 0.5 mm and/or less than 5 mm. R3 is typically less than the radius R1 of guide tube 22, and is further typically at least 30% and/or less than 60% (e.g., between 30% and 60%, e.g., 50%), of R1. Alternatively or additionally, a radius R2 of distal end 16, measured at a distance D1 of 3 mm from distalmost point 45, is at least 1 mm and/or less than 2 mm.

FIG. 1 also shows sheath 60, which is sized and shaped to surround the guide tube. Typically, a diameter D0 of sheath 60 is at least 6 mm and/or less than 15 mm.

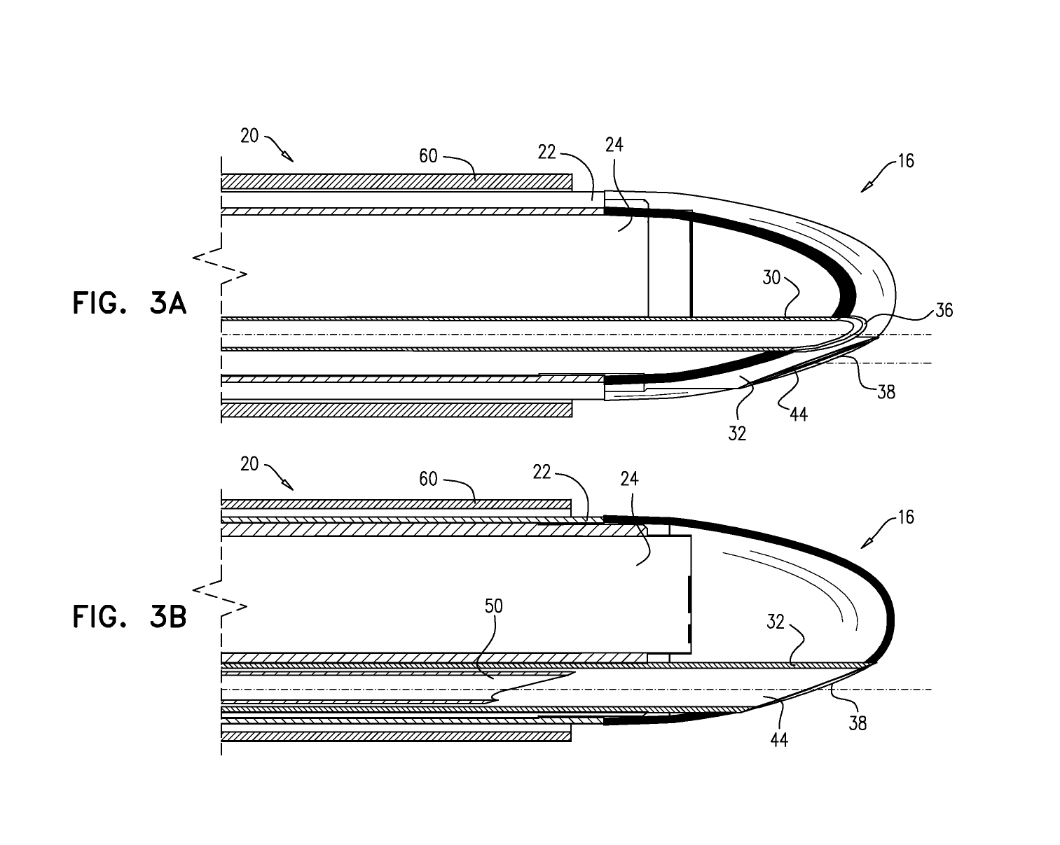

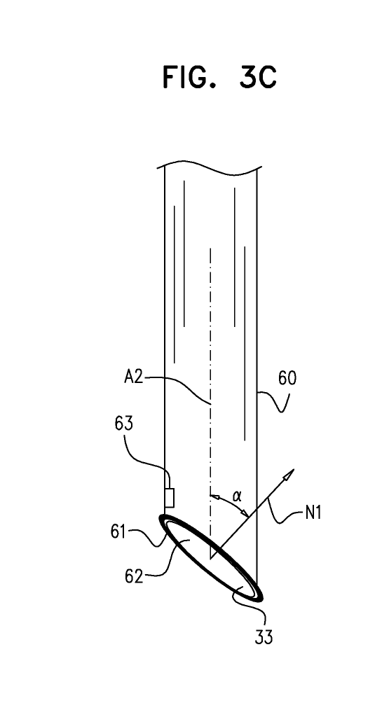

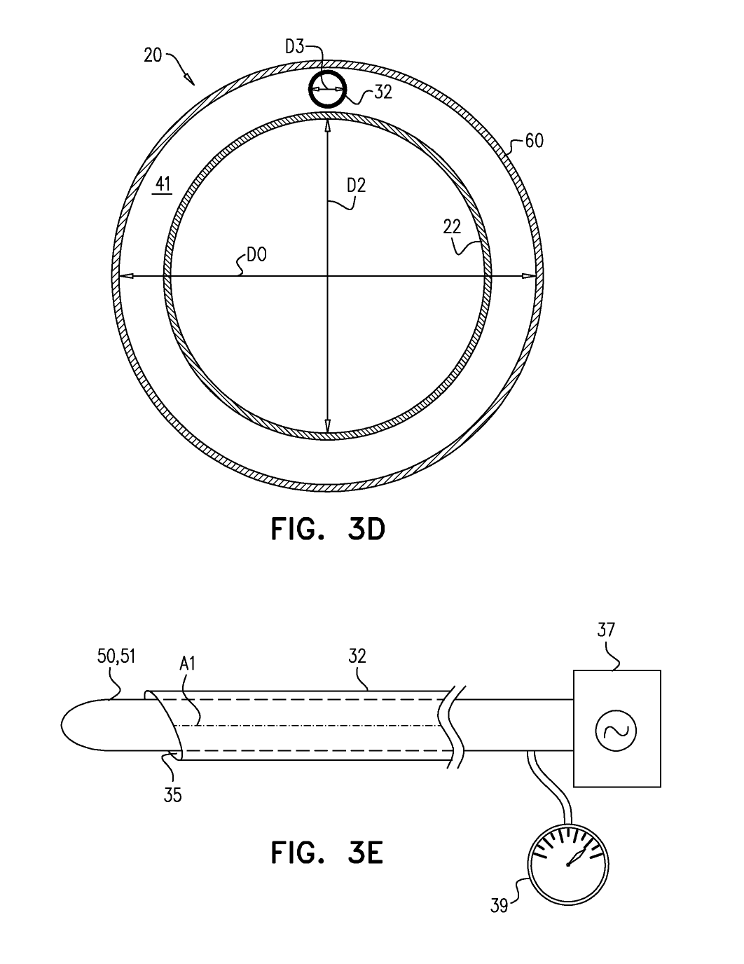

Reference is now made to FIG. 2A, which is a schematic illustration of apparatus 20, in accordance with some applications of the present invention. Reference is also made to FIGS. 3A-B, which are longitudinal cross-sections of apparatus 20, in accordance with some applications of the present invention.

In addition to having distal end 16, guide tube 22 has a proximal end 14, and is shaped to define a guide-tube lumen 18 between proximal end 14 and distal end 16. A diameter D2 (FIG. 1) of guide-tube lumen 18 is typically greater than 4 mm and/or less than 15 mm.

Typically, at least part of outer surface 17 of distal end 16 is transparent. Typically, apparatus 20 comprises an imaging device 24, e.g., a camera, disposed at least partially at distal end 16, and the transparency of distal end 16 facilitates the use of imaging device 24. In some applications, the imaging device comprises an imaging sensor that is disposed within the guide tube (e.g., within guide-tube lumen 18 and/or distal end 16). Alternatively or additionally, imaging device 24 comprises a fiber optic array having a distal end that is disposed within the guide tube, and an imaging sensor coupled to a proximal end of the fiber optic array (application not shown). For some applications, at least part of imaging device 24 is disposed within 15 mm of distalmost point 45 of distal end 16.

Typically, at least one illumination-providing element 26 is disposed at least partially within the guide tube and is configured to provide visible and/or infrared illumination for operation of the imaging device. (For example, four illumination-providing elements may be disposed in the guide tube, as shown in FIG. 2A.) In some applications, illumination-providing element 26 comprises a light source (e.g., a light emitting diode (LED)) disposed within the guide tube. Alternatively or additionally, the illumination-providing element comprises an optical fiber having a distal end that is disposed within the guide tube, and a light source coupled to a proximal end of the optical fiber (application not shown).

Apparatus 20 is typically configured such that most of the light that is emitted from the illumination-providing element is not directly reflected to the imaging device by distal end 16, as such reflection might cause the imaging device to be at least partially “blinded”. Typically, at least 80% of light that is emitted from the at least one illumination-providing element and reflected by distal end 16 is not directly reflected to the imaging device. In some applications, this property of apparatus 20 is at least partially due to a disposition of the at least one illumination-providing element with respect to the imaging device. Alternatively or additionally, a shape of the distal end 16, and/or an optical parameter of distal end 16 and/or of coating applied internally or externally to distal end 16, may facilitate the relatively small amount of blinding reflection. Alternatively or additionally, the relatively small amount of blinding reflection may be facilitated by one or more polarizing filters (not shown) that at least partially cover the at least one illumination-providing element, and/or the imaging device, and/or an inner surface of distal end 16.

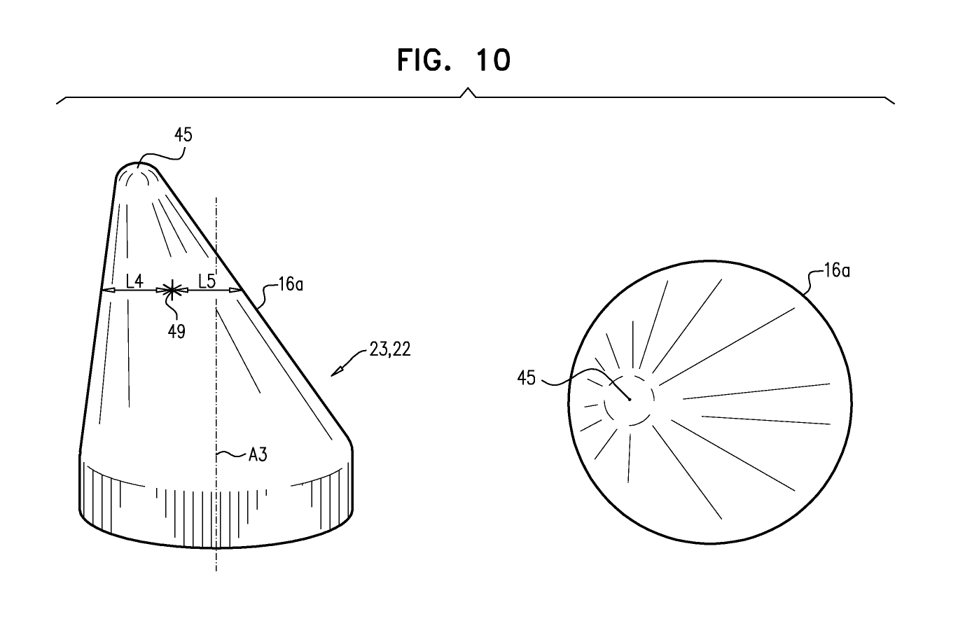

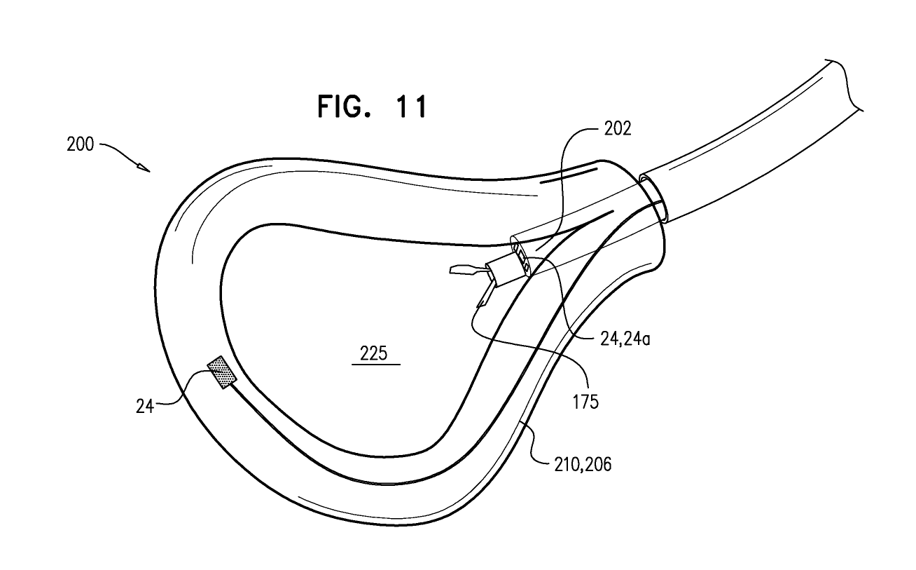

Reference is now additionally made to FIG. 10, which is a schematic illustration of an asymmetric distal end 16a of longitudinal guide member 23 (e.g., guide tube 22), in accordance with some applications of the present invention. As noted above, in some applications, the shape of distal end 16 may facilitate the relatively small amount of blinding reflection. For example, distal end 16a may be rotationally asymmetric with respect to a local central longitudinal axis A3 of the guide tube, such that, for example, at at least one site within 1 mm of distalmost point 45 of distal end 16a, and/or at all sites within 3 mm of the distalmost point of the distal end, a centroid 49 of a cross-section of the distal end does not lie on axis A3. (In FIG. 10, the distances L4 and L5 are equal, i.e., the marked point is indeed centroid 49.)

Returning to FIG. 2A, in some applications, the illumination is changed dynamically, depending on a level of glare that is ascertained in the image. (The level of glare in the image corresponds to the amount of blinding reflection referred to above.) For example, illumination-providing element 26 may be moved (e.g., along a track 27) in response to an ascertained level of glare, in order to potentially reduce the glare. Alternatively or additionally, in applications in which at least two (i.e., first and second) illumination-providing elements are used, one illumination-providing element may be turned off, and another turned on, in response to the ascertained level of glare, in order to potentially reduce the glare. In other words, in response to the level of glare, exactly one illumination-providing element is selected from the first illumination-providing element and the second illumination-providing element, and illumination is provided from the selected illumination-providing element. For example, in response to the level of glare, first LED 26a may be lit, while second LED 26b is not lit. Typically, the glare-reduction techniques described herein are performed automatically by a processor.

The imaging sensor belonging to imaging device 24, described hereinabove, may be considered to be a heart-proximity sensor, in that the imaging performed by imaging device 24 helps the operator ascertain that distal end 16 is proximate to the heart; typically, imaging device 24 is used to generate an image of at least part of the heart of the subject, and the proximity is ascertained, using the image. Alternatively or additionally to imaging device 24, apparatus 20 comprises one or more other types of heart-proximity sensor 29 configured to generate a signal indicative of a proximity of the distal end of the guide tube to the heart of the subject. Before deploying the suction port of the sheath over the guide tube, heart-proximity sensor 29 is used to generate the signal, and the proximity is ascertained, using the signal. As shown in FIG. 2A, heart-proximity sensor 29 is typically coupled to distal end 16, e.g., to outer surface 17 of distal end 16.

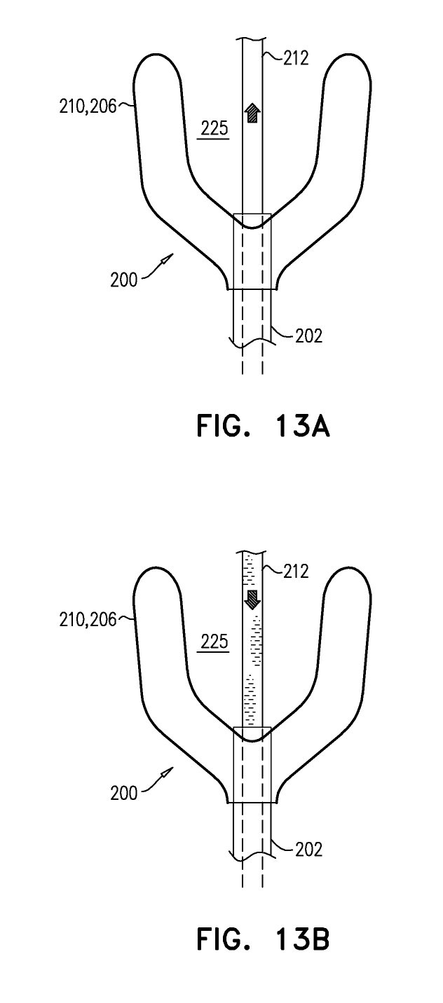

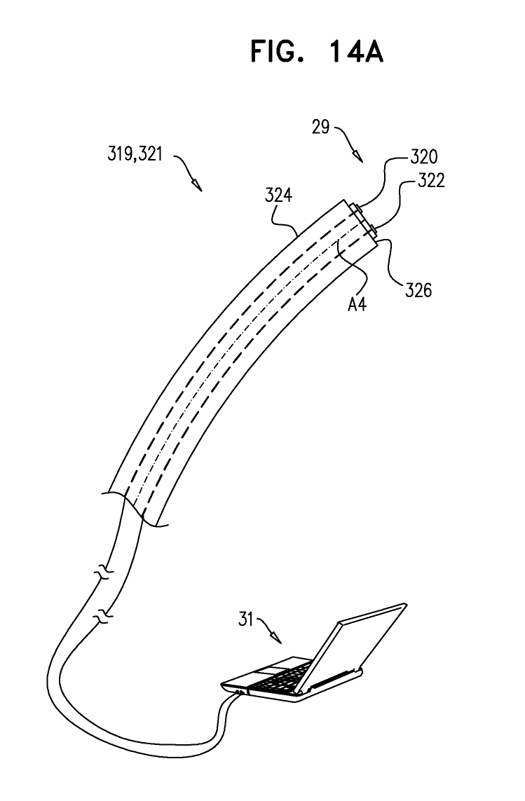

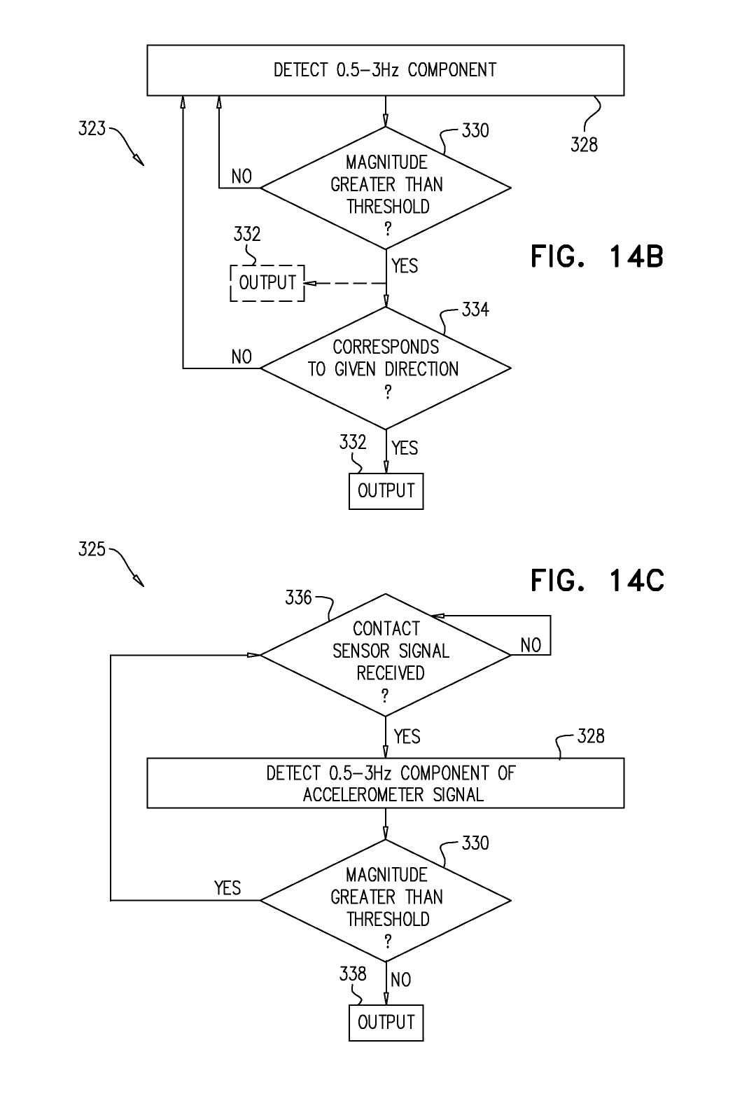

In some applications, heart-proximity sensor 29 comprises an ultrasound (e.g., a Doppler ultrasound) sensor (e.g., transducer). In response to a signal (e.g., an ultrasound image) received by the ultrasound sensor, the operator may ascertain that distal end 16 is proximal to the heart. (The ultrasound transducer may also be used for treatment.) Alternatively or additionally, the heart-proximity sensor comprises a contact sensor configured to generate a signal indicative of contact of the blunt distal end of the guide tube to the heart of the subject. Alternatively or additionally, the heart-proximity sensor comprises an accelerometer. Typically, for applications in which an accelerometer is used, apparatus 20 further comprises a processor 31 configured to generate an output indicative of the proximity of the blunt distal end of the guide tube to the heart of the subject, based on a signal received from the accelerometer, as further described immediately hereinbelow with reference to FIGS. 14A-C.

While advancing guide tube 22 toward the heart of the subject, care must be taken not to damage (e.g., puncture) internal organs, such as the liver, of the subject. In some applications, the heart-proximity sensor comprises both an accelerometer and a contact sensor, which together help prevent such damage from occurring, as further described immediately hereinbelow with reference to FIGS. 14A-C.

The scope of the present invention includes use of certain proximity-sensing apparatus and methods, even outside of the context of pericardium penetration. In this regard, reference is now additionally made to FIG. 14A, which is a schematic illustration of proximity-sensing apparatus 319 and 321, and to FIGS. 14B-C, which are flow charts of proximity-sensing methods 323 and 325, in accordance with some applications of the present invention. The description below of these figures is also applicable to apparatus 20, mutatis mutandis.

FIG. 14 shows apparatus 321, in which processor 31 is configured to generate an output indicative of the proximity of the distal portion 326 of a catheter 324 to the heart of the subject. Proximity-sensing method 323, performed in combination with apparatus 321 (and, as noted above, apparatus 20), is depicted in FIG. 14B. Apparatus 321 comprises an accelerometer 320, which generates a signal upon being accelerated. In a detection step 328, processor 31 detects a component of the signal having a frequency between 0.5 and 3 Hz, which generally encompasses the typical range of heartbeat frequencies. In a magnitude-comparison step 330, the magnitude of this component is compared to a threshold. If the magnitude is greater than the threshold, the processor may generate the output in output step 332. (This step is indicated by the dashed arrow and box in FIG. 14B.) The output may include audio and/or visual output to the operator of the apparatus, indicating proximity to the heart.

In some applications, directional accelerometry is used, and the processor is configured to generate the output only if (per a direction-comparison step 334) the detected component of the signal corresponds to a direction that is generally perpendicular to a local central longitudinal axis A4 of the catheter. In the context of apparatus 20, the processor generates the output only if the detected component of the signal corresponds to a direction that is generally perpendicular to a plane 33 (FIG. 3C) defined by suction port 62 of the sheath, and/or is generally parallel to a central longitudinal axis of the guide tube at the distal end of the guide tube (cf. A3 in FIG. 10); thus, the operator receives confirmation that apparatus 20 is aligned properly with respect to the heart.

FIG. 14 also shows apparatus 319 comprising a heart-proximity sensor 29 disposed at distal portion 326 of catheter 324. Heart-proximity sensor 29 comprises accelerometer 320 and a contact sensor 322, which work together to generate a signal indicative of a proximity of distal portion 326 to an internal organ of a subject that is not the heart of the subject. (In other words, the signals from contact sensor 322 and accelerometer 320 together make up a signal that is indicative of contact with the internal organ that is not the heart.) As noted above, this signal may help prevent damage to the internal organ. Proximity-sensing method 325, depicted in FIG. 14C, is practiced in combination with apparatus 319, and, as noted above, with apparatus 20.

If contact sensor 322 contacts the organ, the contact sensor generates a signal that is indicative of this contact. The signal from the contact sensor is received, e.g., by processor 31, in a contact-signal-receiving step 336. Then, detection step 328, as described above with reference to FIG. 14B, is executed, e.g., by processor 31. If, per magnitude-comparison step 330, the magnitude of the detected 0.5-3 Hz component exceeds the threshold, it is likely that contact has been made with the heart, and thus, no output is generated. If, on the other hand, the magnitude does not exceed the threshold, it is likely that contact has been made with an internal organ that is not the heart. In response, processor 31 may generate an output, in an output step 338. The output may take the form of an audio and/or visual warning to the operator to change the advancement trajectory.

Apparatus 319 and 321 may be combined into a single apparatus, and methods 323 and 325 may be practiced in combination with each other. For example, method 325 may include direction-comparison step 334 of method 323, prior to output step 338.

Returning to FIG. 2A, apparatus 20 further comprises a puncturing element 50, e.g., a needle 51 shaped to define a lumen thereof. As described hereinabove with reference to FIGS. 5A-E, puncturing element 50 is configured to puncture a portion of the pericardium while the portion of the pericardium is in sheath 60. In some applications, apparatus 20 further comprises a puncturing-element tube 32, and puncturing element 50 is sized and shaped to be passable through puncturing-element tube 32 and out of a distal end of the puncturing-element tube. In some applications, as shown in FIG. 2A, distal end 16 (e.g., outer surface 17) is shaped to define a puncturing-element-tube hole 44 therein, and puncturing-element tube 32 is disposed within the guide tube (e.g., within lumen 18) such that the puncturing-element tube is in contact with a perimeter 38 of puncturing-element-tube hole 44. For example, a distalmost perimeter of puncturing-element tube 32 may contact perimeter 38. Typically, a diameter of puncturing-element-tube hole 44 is at least 0.2 mm and/or less than 2 mm; alternatively, the diameter is at least 2 mm and/or less than 5 mm. (In the context of the present description and claims, the term “diameter,” when applied to an ellipse, refers to a mean of the lengths of the major and minor axes. Puncturing-element-tube hole 44 is elliptically shaped in FIG. 2A, such that the diameter of the hole is the mean of lengths L2 and L3.)

Reference is now additionally made to FIG. 3D, which is a cross-section of apparatus 20, in accordance with some applications of the present invention. In some applications, puncturing-element tube 32 is disposed between sheath 60 and guide tube 22, instead of within the guide tube. In some applications, puncturing element 50 is not disposed inside of puncturing-element tube 32, but rather, is disposed directly inside of guide-tube lumen 18 or between guide member 23 (e.g., guide tube 22) and sheath 60.

Reference is now additionally made to FIG. 3E, which is a schematic illustration of puncturing-element tube 32, in accordance with some applications of the present invention. In some applications, a distal end of puncturing-element tube 32 defines a face 35 that is not perpendicular to a local central longitudinal axis A1 of puncturing-element tube 32. (In the context of the present description and claims, the “central longitudinal axis” of a structure refers to the set of centroids of all cross-sections of the structure. The central longitudinal axis may curve, such that the orientation of the central longitudinal axis varies across its length; the “local” central longitudinal axis refers to the tangent to the central longitudinal axis at or generally near the point of interest.)

As further shown in FIG. 3E, in some applications, puncturing element 50 comprises a radiofrequency wire and/or a radiofrequency needle 51, and apparatus 20 further comprises a radiofrequency generator 37 configured to transmit a radiofrequency signal to a distal end of the radiofrequency wire and/or needle. The radiofrequency signal facilitates the puncture of the pericardium.

In some applications, apparatus 20 further comprises a sensor 39 configured to measure an electrophysiological signal occurring at a tip of puncturing element 50. For example, a voltmeter coupled to a proximal end of the puncturing element may measure a voltage occurring at the tip of the puncturing element. Since the voltage within the pericardium is different from the voltage outside of the pericardium, sensor 39 may help the operator ascertain that the pericardium has been punctured.

As described hereinabove with reference to FIGS. 5A-E, apparatus 20 is configured to facilitate drawing a portion of a pericardium of the heart through suction port 62 of sheath 60 and into the sheath, by the application of suction. In some applications, puncturing-element tube 32 is a puncturing-element-and-suction tube, i.e., suction may be applied through the puncturing-element tube, in order to draw in the portion of the pericardium. Alternatively or additionally, as shown in FIG. 2A, apparatus 20 comprises a separate suction tube 30, configured to facilitate the drawing in of the portion of the pericardium by the application of suction through suction tube 30.

In some applications, as shown in FIG. 2A, distal end 16 is shaped to define a suction-tube hole 42 therein, and the suction tube is disposed within the guide tube (e.g., within lumen 18) such that a distal end of the suction tube is in contact with a perimeter 36 of the suction-tube hole. Suction tube 30 facilitates the application of suction through suction-tube hole 42 to draw the portion of the pericardium into sheath 60. A diameter of suction-tube hole 42 is typically at least 0.2 mm, e.g., at least 0.5 mm, and/or less than 2 mm; alternatively, the diameter is at least 2 mm and/or less than 5 mm. (A “diameter” of an ellipse is defined above.) In other applications, suction tube 30 is disposed between the guide tube and the sheath, as shown in FIG. 3D for the puncturing-element tube.

For some applications, a distal end of suction tube 30 defines a face that is not perpendicular to a local central longitudinal axis of suction tube 30, as shown in FIG. 3D for puncturing-element tube 32.

In some applications, suction is applied through a space 41 (FIG. 3D) between an outer wall of guide tube 22 and an inner wall of sheath 60, alternatively or additionally to the application of suction through suction tube 30. In such applications, space 41 is typically large enough to facilitate the application of suction, but not so large as to have an unduly-large diameter of the sheath. For example, diameter D0 of sheath 60 may be greater than 0.1 mm (e.g., greater than 0.2 mm) and/or less than 4 mm (e.g., less than 0.6 mm) greater than a diameter D2 of the guide tube.

In some applications, an oscillating suction pressure is applied, such as to facilitate separation of the portion of the pericardium from the tissue (e.g., myocardial tissue) that is underneath it. Alternatively or additionally, to facilitate this separation, a suction pressure that increases at an average rate of at least 5 and/or less than 15 mm Hg per second is applied for at least 1 second. In some applications, apparatus 20 comprises a vibrating element 47 configured to vibrate the distal end of the guide tube during and after the drawing of the portion of the pericardium into sheath 60. This vibration may help separate the portion of the pericardium from the portion of the myocardium that is underneath it.

In some applications, rinsing fluid may be passed through puncturing-element tube 32 and puncturing-element-tube hole 44, and/or through suction tube 30 and suction-tube hole 42, in order to remove debris from an external surface of distal end 16. In some applications, the rinsing fluid is passed through a separate rinsing-fluid lumen in guide tube 22 (not shown), and/or through a separate hole in distal end 16 (not shown).

In some applications, the advancement of apparatus 20 is facilitated by the use of electrophysiological sensing. For example, electrodes may be attached to apparatus 20 (e.g., to distal end 16 and/or puncturing element 50), the electrodes electrically coupled to an extracorporeal monitor. The electrodes facilitate navigation of apparatus 20 by detecting electrical activity of the heart (e.g., ECG signals). Alternatively or additionally, such electrodes may be radiopaque, and may facilitate navigation of apparatus 20 toward the heart by use of fluoroscopic imaging techniques.

Other navigation techniques include use of a 3D (i.e., position only) or 6D (i.e., position and orientation) navigation system in order to facilitate safe and efficient access to the heart. In some applications, a position sensor 43 disposed at a distal portion of the guide tube (e.g., coupled to distal end 16) is used to measure a position of distal end 16, and a proximity of the distal end of the guide tube to the heart of the subject is ascertained, using the measured position. In some applications, sensor 43 is a position-and-orientation sensor, and the position and orientation measured by sensor 43 are used to navigate the guide member. Alternatively or additionally, sensor 43 comprises an ultrasound sensor (e.g., a Doppler ultrasound sensor), and the image from the ultrasound sensor is used to navigate the guide tube. In some applications, sensor 43 is integrated with a CARTO™ or NavX™ navigation system.

For some applications, a preoperative image (e.g., a preoperative CT image) of the subject is used for navigation. The preoperative image may be used in combination with the 3D or 6D navigation system described above, and/or in combination with realtime images from imaging device 24, e.g., via use of image registration techniques.

Typically, a handle 12, shown in FIG. 2A, facilitates the advancement and operation of apparatus 20.

Reference is now made to FIG. 2B, which is a schematic illustration of apparatus 21 for penetrating the pericardium, in accordance with some applications of the present invention. Apparatus 21 is generally similar to apparatus 20. A notable difference, however, is that distal end 16 of guide member 23 is not closed, as further described hereinbelow.

Apparatus 21 comprises guide member 23 (e.g., guide tube 22). As in apparatus 20, guide tube 22 has a proximal end 14 and a distal end 16, and is shaped to define a guide-tube lumen 18 between the proximal and distal ends. A distal-end cover 116 is shaped to (i) define an aperture 100 therein, and (ii) cover distal end 16 of the guide tube. Distal-end cover 116 is typically shaped such as to facilitate blunt dissection and safe navigation and advancement of apparatus 21 toward the heart, as described hereinabove with respect to distal end 16 of apparatus 20. Aperture 100 typically has a diameter D4 of at least 1 mm and/or less than 5 mm. In some respects, the function of distal-end cover 116 of apparatus 21 is analogous to that of distal end 16 of apparatus 20. For example, heart proximity sensor(s) 29 (FIG. 2A) may be coupled to distal-end cover 116, and may be used to help ascertain proximity to and/or contact with the heart or another internal organ, as described hereinabove.

Imaging device 24 is typically disposed at distal end 16 of guide tube 22. Apparatus 21 may further comprise one or more, e.g., four, illumination-providing elements 26, e.g., LEDs, as describe hereinabove with reference to apparatus 20. In some applications, distal-end cover 116 is transparent. In other applications, distal-end cover 116 is not transparent; in such applications, imaging device 24 is typically aligned with aperture 100, and illumination-providing elements 26 are typically disposed such that illumination may pass through aperture 100. (Typically, apparatus 21 is configured to reduce blinding reflections, as described hereinabove with respect to apparatus 20.)

As in apparatus 20, puncturing element 50 is disposed within guide-tube lumen 18, or between guide member 23 (e.g., guide tube 22) and sheath 60. In some applications, puncturing element 50 is disposed within puncturing-element tube 32, as in apparatus 20. FIG. 2B shows an application in which puncturing-element tube 32 extends through lumen 18 and contacts perimeter 38 of puncturing-element-tube hole 44 in distal end 16. In such applications, distal-end cover 116 is typically shaped to define a second puncturing-element-tube hole 144 in alignment with hole 44, for passage therethrough of puncturing element 50. In some applications, apparatus 21 includes a handle 112, which facilitates the advancement and operation of the apparatus.

The lens 25 of imaging device 24 might become obstructed by debris that enters aperture 100. Hence, in some applications, apparatus 21 includes elements that facilitate cleaning of the lens. For example, distal end 16 may be shaped to define a rinse port 136 therein, rinse port 136 typically being close to lens 25 of imaging device 24. In such applications, apparatus 21 further comprises a rinse tube 130 in fluid communication with the rinse port, e.g., in contact with a perimeter 137 of rinse port 136. Rinse fluid may be passed through rinse tube 130 and through port 136 to remove the debris, thus facilitating the imaging functionality of imaging device 24. In some applications, distal end 16 is further shaped to define a groove 142, which facilitates the flow of the rinse fluid over the lens of the imaging device.

In some applications, rinse tube 130 is a rinse-and-suction tube, i.e., it also functions as a suction tube, for facilitating drawing the portion of the pericardium into the sheath. In other applications, apparatus 21 comprises a separate suction tube, e.g., as described hereinabove with respect to apparatus 20.

In general, the description herein relating to the operation of apparatus 20, e.g., with respect to the drawing of the sheath over the guide tube, puncturing of the pericardium, etc. also relates to apparatus 21, mutatis mutandis.

Reference is now made to FIG. 2C, which is a schematic illustration of apparatus 21′ for penetrating the pericardium, in accordance with some applications of the present invention. Apparatus 21′ differs from apparatus 21, in that guide member 23 is a shaft 55 shaped to define one or more longitudinal channels 57 therealong, instead of guide member 23 being guide tube 22. (In order to show shaft 55, sheath 60 is hidden from view in FIG. 2C.) Shaft 55 may also be used instead of guide tube 22 in apparatus 20, yielding alternate apparatus 20′. In general, apparatus 20′ is functionally equivalent to apparatus 20, and apparatus 21′ is functionally equivalent to apparatus 21. Generally, longitudinal channel(s) 57 take the place of guide-tube lumen 18. For example, puncturing element 50, puncturing-element tube 32, rinse tube 30, etc. may all be disposed within one or more longitudinal channels in shaft 55. The entire description herein of apparatus 20 and apparatus 21 Is intended to also relate to apparatus 20′ and apparatus 21′, even when guide tube 22 is specifically mentioned. (Distal-end cover 116 is not shown in FIG. 2C.)

Reference is now made to FIGS. 2D-E, which are schematic illustrations of apparatus 300 for penetrating a pericardium, in accordance with some applications of the present invention. Apparatus 300 comprises longitudinal guide member 23, i.e., guide tube 22 or shaft 55, comprising a distal end 302. Distal end 302 differs from distal ends 16 and 16a described hereinabove with reference to apparatus 20 and 21, respectively, as further described hereinbelow. Another difference between apparatus 300 and apparatus 20/21 is that apparatus 300 does not necessarily include sheath 60, as further described hereinbelow. (In most other respects, apparatus 300 is generally similar or identical to apparatus 20 and/or apparatus 21.)

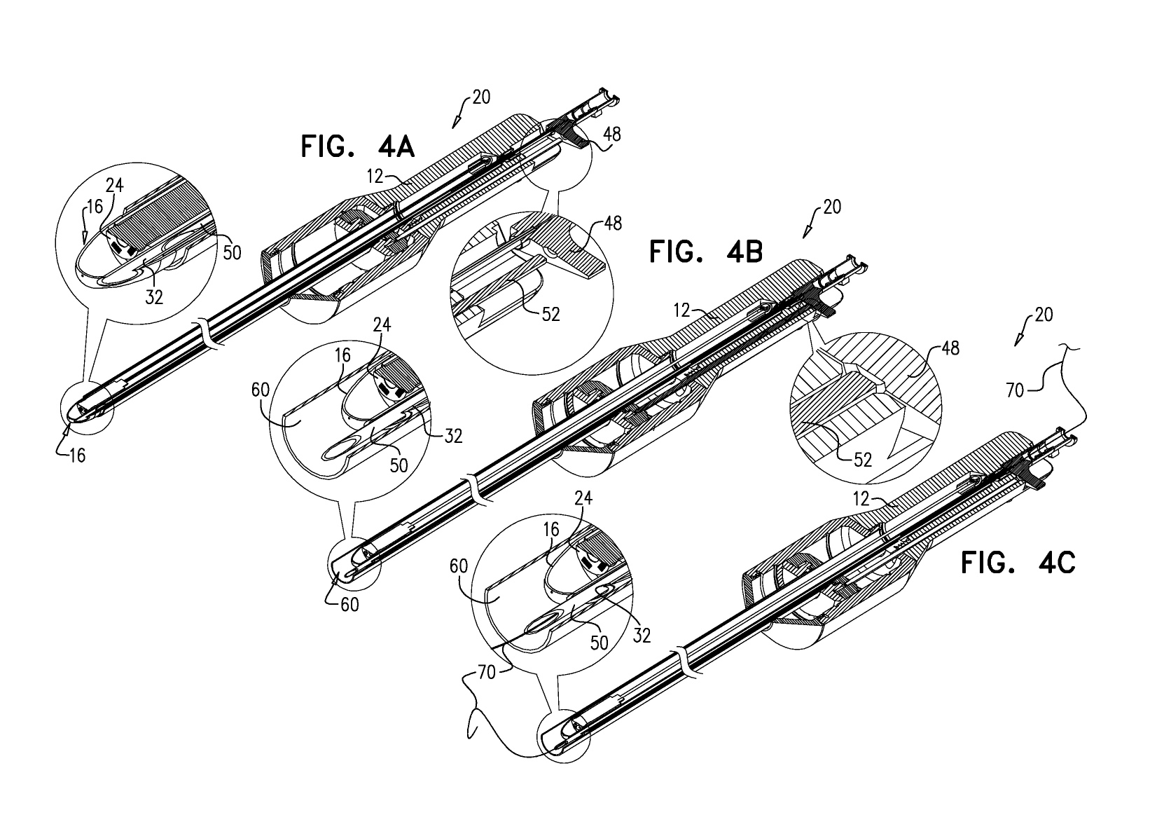

As shown in FIGS. 2D-E, distal end 302 comprises an outer tube-wall 304 shaped to define a lumen 306 thereof, and an inner tube-wall 308 disposed within lumen 306. Apparatus 300 is configured to facilitate the drawing of the portion of the pericardium into the portion of lumen 306 that is between outer tube-wall 304 and inner tube-wall 308, by application of suction through the portion of the lumen. For example, suction may be applied directly through the lumen, and/or via suction tube 30. Apparatus 300 also comprises puncturing element 50, which is configured to puncture the portion of the pericardium while the portion of the pericardium is in the portion of lumen 306 that is between outer tube-wall 304 and inner tube-wall 308. Typically, puncturing element 50 is disposed such that a distal end of the puncturing element passes between the two tube-walls. Apparatus 300 also comprises puncturing-element-restraining element 52, described hereinbelow with reference to FIGS. 4A-C.

In general, outer-tube wall 304 functions in a similar manner to sheath 60, at least in that it provides a suction port 310 through which the portion of the pericardium may be drawn. Thus, in most (but not necessarily all) applications, apparatus 300 does not include sheath 60.

Typically, distal end 302 further comprises a blunt dome-shaped cover 312, at least part of which is transparent. Cover 312 is disposed at a distal end of inner tube-wall 308 and covers space that is inside the inner tube-wall. The transparency of cover 312 facilitates imaging by imaging device 24, and the bluntness of cover 312 facilitates safe and effective advancement of guide member 23 toward the heart, as described hereinabove with respect to outer surface 17 of distal end 16 (FIG. 1). Furthermore, cover 312 helps keep the portion of the pericardium to which suction is applied from enveloping outer tube-wall 304, by applying a counteracting force to the suction.

Typically, the inner diameter D3 of outer tube-wall 304 is at least 0.2 mm and/or less than 4 mm greater than the outer diameter D5 of inner tube-wall 308. (This allows for a space between the two walls that is large enough to facilitate the application of suction, but not so large as to have an unduly-large outer diameter of outer tube-wall 304.) In some applications, the distalmost perimeter 314 of the inner tube-wall is at least 0.2 mm and/or less than 4 mm distal to the distalmost perimeter 316 of the outer tube-wall, such that, for example, height H shown in FIG. 2D is between 0.2 and 4 mm. In general, the greater distal reach of inner tube-wall 308 helps keep the portion of the pericardium to which suction is applied from enveloping outer tube-wall 304, as described hereinabove with respect to cover 312. (In particular, the greater distal reach of inner tube-wall 308 is helpful for applications in which apparatus 300 does not comprise cover 312.)

Reference is now made to FIGS. 4A-C, which are longitudinal cross-sections of apparatus 20, further showing a puncturing-element-restraining element 52, in accordance with some applications of the present invention. When puncturing the portion of the pericardium within sheath 60 (FIG. 5D), it is generally preferred that puncturing element 50 not pass out of the distal end of the sheath, in order to reduce any potential damage to the heart. In order to inhibit passage of puncturing element 50 out of sheath 60, apparatus 20 comprises puncturing-element-restraining element (e.g., a needle-restraining element) 52 shaped and positioned with respect to the puncturing element to inhibit passage of a distal tip of the puncturing element out of a distal end of the sheath. FIGS. 4A-C show needle-restraining element 52 as a rod-shaped element by way of illustration and not limitation.

As described hereinabove, apparatus 20 may comprise puncturing-element tube 32, e.g., disposed within guide tube 22. Advancement of puncturing element 50 (e.g., needle 51) distally in puncturing-element tube 32 is shown in FIG. 4B by way of illustration and not limitation. In FIG. 4B, a needle handle 48 is pushed distally to advance needle 50 within tube 22 and subsequently out of tube 22 and into the area surrounded by sheath 60. Additionally, movement of handle 48 in a distal direction engages needle-restraining element 52 with needle handle 48, as shown in FIG. 4B, to inhibit passage of needle 50 out of sheath 60. Thus, apparatus 20 is configured to reduce the possibility of puncturing element 50 injuring myocardial tissue 93 (shown in FIGS. 5A-5E). Needle-restraining element 52 may also be used for applications in which apparatus 20 does not comprise puncturing-element tube 32.

Reference is now made to FIG. 3C, which is a schematic illustration of sheath 60 and suction port 62, in accordance with some applications of the present invention. As described hereinabove with reference to FIGS. 5A-E, sheath 60 is shaped to define an at least partially distally-facing suction port 62 at the distal end of the sheath, and apparatus 20 is configured to facilitate drawing a portion of a pericardium 90 of the heart through the suction port and into the sheath. Suction port 62 defines a plane 33 that may take on various orientations with respect to the local central longitudinal axis A2 of the sheath. Typically (although not always), the preferred orientation is a function of the angle theta (FIG. 1) at which the apparatus is advanced toward the heart. In some applications, such as those involving a relatively shallow approach of the apparatus toward an anterior portion of the heart the angle alpha between (a) axis A2, and (b) a normal N1 to plane 33, is between 40 and 70 degrees. In other applications, such as those involving a relatively steep approach of the apparatus toward a posterior portion of the heart, angle alpha is at least 0 degrees (e.g., at least 10 degrees) and/or less than 50 degrees (e.g., less than 40 degrees). Given that suction port 62 is at least partially distally-facing, alpha is always less than 90 degrees. (Most of the figures, e.g., FIG. 1, show a completely distally-facing suction port, i.e., an angle alpha of 0.)

Reference is also made to FIG. 5B. Typically, a line L1 that is 4 mm, 1 cm, or 1.5 cm long and extends from a center of suction port 62 into the sheath, in a direction parallel to axis A2, does not contact any part of apparatus 20, when suction port 62 is distal to the guide tube. (Thus, there is generally enough space in the sheath for the portion of the pericardium that is drawn in, and the tip of puncturing element 50 is generally kept at a safe distance from tissue 93 that is underneath the pericardium.) In some applications, an o-ring 61 is disposed at the suction port of the sheath, e.g., in order to help seal the interface between the suction port and the pericardium.

For some applications, at least one force sensor 63 is coupled to an inner surface of sheath 60. Force sensor 63 generates a force sensor signal responsive to contact of pericardium 90 with the inner surface of sheath 60, to determine, responsive to the signal, the degree to which the pericardium has been drawn in to sheath 60.

For some applications, portions of apparatus 20 are transparent to X-ray, to allow X-ray-based imaging techniques (including fluoroscopy) to be used to assist in navigating the apparatus. For example, outer surface 17 of distal end 16 is typically transparent to X-ray. In some applications, at least a distal portion of sheath 60 (e.g., a portion extending proximally at least 1 cm from the distal end of sheath 60) is transparent to X-ray to enable X-ray imaging of the drawing of the pericardium into sheath 60. For some such applications, needle 50 injects a contrast medium prior to the pericardium being drawn into sheath 60, which allows the drawing of the pericardium into sheath 60 to appear on X-ray. Additionally or alternatively, needle 50 injects a contrast medium into the pericardial region subsequently to puncturing the pericardium, in order to allow X-ray imaging of the heart and pericardial region.

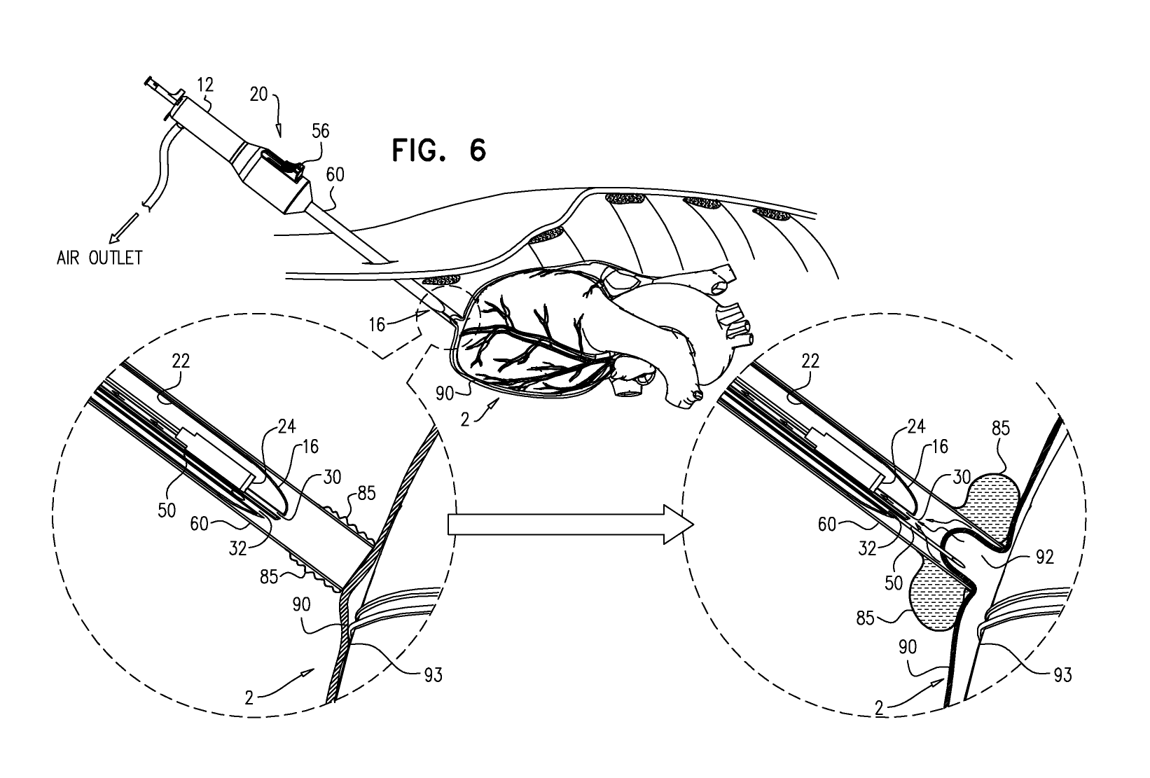

Reference is now made to FIG. 6, which is a schematic illustration of an inflatable element 85 (e.g., a balloon) for use with apparatus 20 and 21. Inflatable element 85 is disposed over sheath 60. Inflation of inflatable element 85 facilitates the gripping of the portion of pericardium 90 by sheath 60, e.g., by increasing the area over which the suction is applied to the portion of pericardium. Element 85 may be inflated prior to puncturing element 50 puncturing the pericardium, such as to facilitate the puncturing, or subsequently to the puncturing, to keep pericardial region 92 from closing.

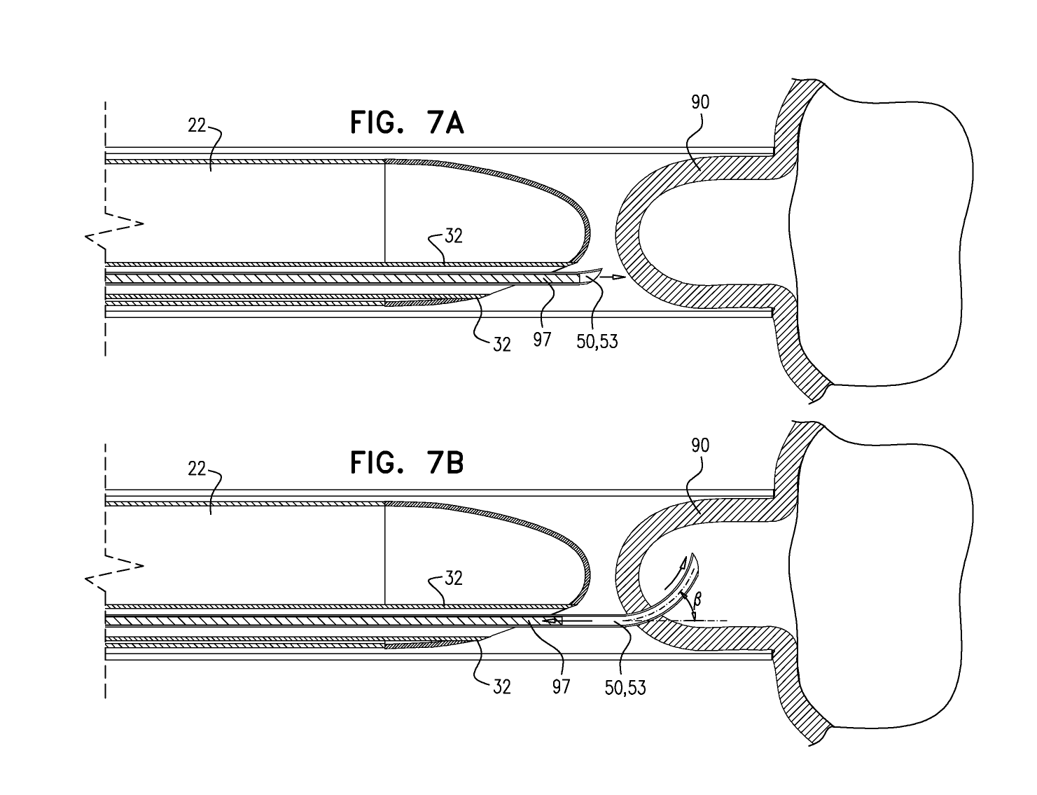

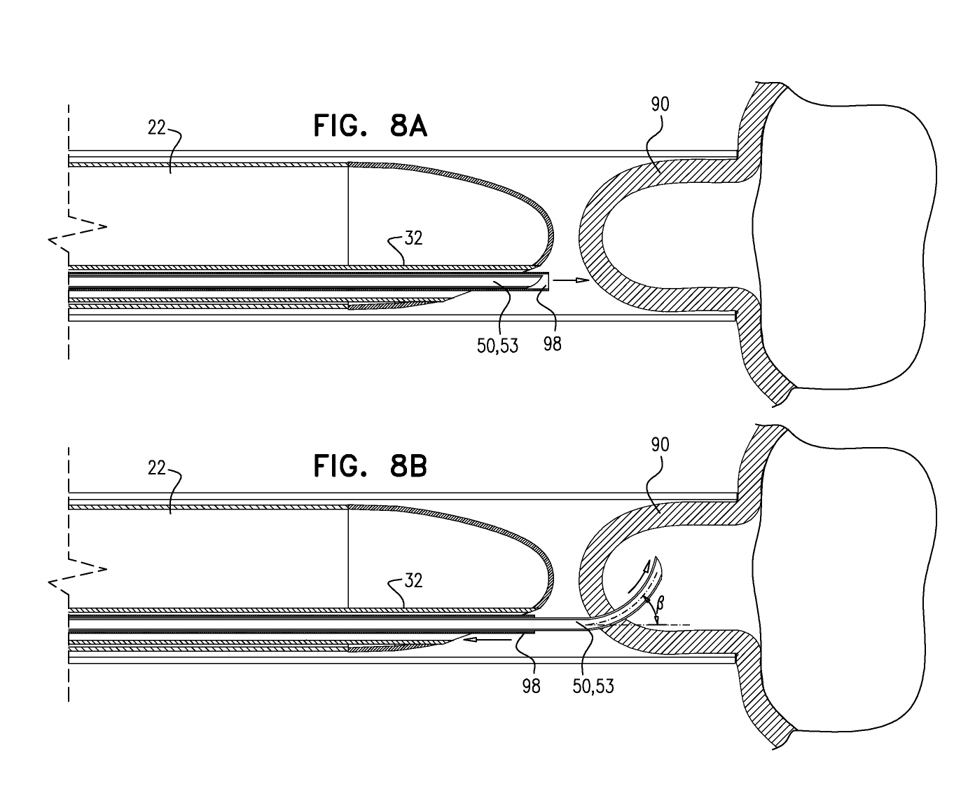

Reference is now made to FIGS. 7A-B and 8A-B, which are schematic illustrations of a curved needle 53 for use with applications of the present invention. For some applications, puncturing element 50 comprises a curved needle 53, in which, for example, a distal end of the needle is “J”-shaped. For some applications, curved needle 53 comprises a shape memory alloy, e.g., nitinol. For some such applications, curved needle 53 is maintained in a straight configuration prior to the puncturing of the pericardium, and assumes a curved configuration prior to puncturing the pericardium.

For some applications (FIGS. 7A-B), a rigid core structure (e.g., a straight rigid stainless steel core) 97 is disposed within curved needle 53 and maintains curved needle 53 in a straight configuration while in puncturing-element tube 32. Prior to the puncturing, curved needle 53 is advanced out of puncturing-element tube 32, typically but not necessarily together with rigid core 97 (FIG. 7A). The rigid core is then pulled back (and/or the needle is advanced forward), such mat needle 50 assumes a curved configuration upon disengagement from the rigid core, and the pericardium is punctured (FIG. 7B).

Alternatively, curved needle 53 is surrounded by a needle casing (e.g., a straight rigid casing) 98 while disposed within puncturing-element tube 32 (FIGS. 8A-B). Casing 98 maintains needle 53 in a straight configuration until needle 53 is released from the casing. When needle 53 is advanced out of puncturing-element 32 (FIG. 8A), the rigid casing typically still surrounds needle 53 and maintains the needle in a straight configuration. Release of needle 53 from casing 98 (FIG. 8B), allows needle 53 to assume a curved configuration when puncturing the pericardium.

In FIGS. 7A-B and 8A-B, it is seen that needle 53 punctures the portion of pericardium within sheath 60 at a non-zero angle beta with respect to the longitudinal direction, rather than longitudinally, as shown in FIG. 6. It is hypothesized that, in some applications, puncturing from the side facilitates “grabbing” of the tissue during the puncture, thereby easing the act of puncturing. Alternatively or additionally, puncturing at a non-zero angle, such that the needle is not moving directly toward myocardial tissue, reduces a likelihood of inadvertent damage to the myocardium.

Although FIGS. 7A-B and 8A-B show curved needle 53 being disposed inside of puncturing-element tube 32, it is noted that core structure 97 and casing 98 may also be used without puncturing-element tube 32.

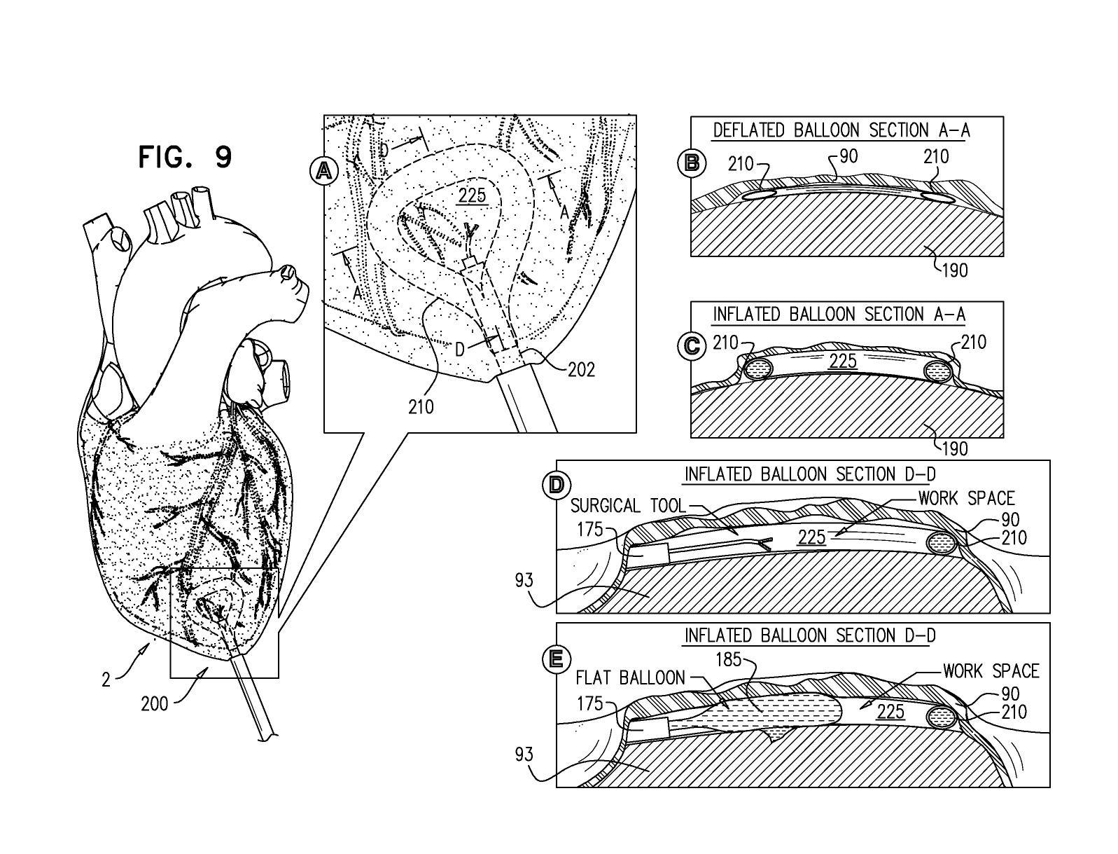

As described hereinabove with reference to FIG. 5E, following the passing of guidewire 70 into pericardial region 92, needle 51 is typically withdrawn, and a tool is then passed over the guidewire and into the pericardial region, as shown in FIG. 9 and described hereinbelow. Alternatively or additionally, a tube (not shown) is passed over the guidewire, the guidewire is withdrawn, and the tool is passed through the tube and into the pericardial region.

In some applications, the tool that is passed into pericardial region 92 includes a reflection-facilitation element, as described, for example, in US 2011/0282249 to Tsoref, which is incorporated herein by reference. As further described in US 2011/0282249 to Tsoref, ultrasound energy may then be transmitted from within a chamber of the heart, toward the reflection-facilitation element, to ablate myocardial tissue.

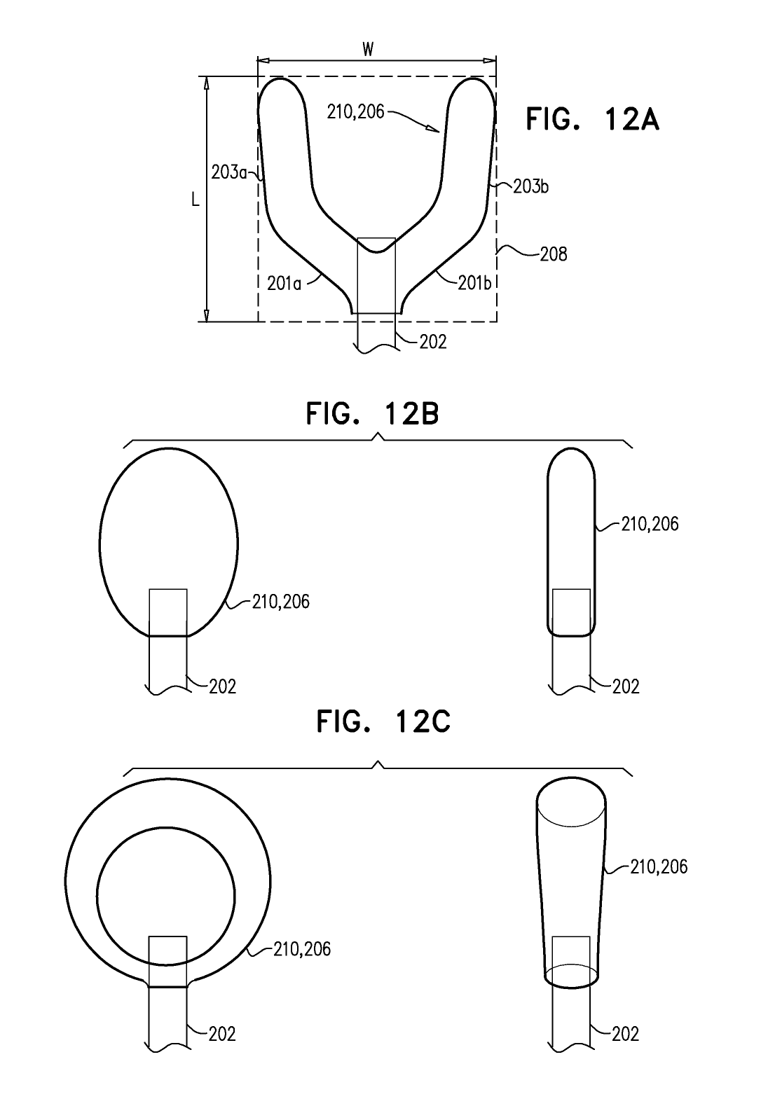

In some applications, as further described hereinbelow with reference to FIGS. 9 and 11, the tool includes an expandable element, e.g., an inflatable element, such as a balloon, and/or an expandable mesh. The pericardial sac is typically lubricated against the beating heart such that navigating within the pericardial region, without the creation of a working space, may be challenging. To at least partially address this challenge, the expandable element may be expanded within pericardial region 92, in order to create a working space. Alternatively or additionally, the expandable element may be used to inhibit bleeding of the heart, by applying pressure.

In applications in which a working space is created, a surgical tool may then be passed over the guidewire and into the working space, in order to perform a surgical procedure on the heart. Creating a working space within a pericardial region, as described in the present application, is useful for facilitating various cardiac procedures, including but not limited to left atrial appendage (LAA) treatment, coronary artery bypass grafts (CABG), and bleeding reduction by application of pressure. Examples of surgical tools that may be used include a forceps, a needle, an electrosurgery tool, a cutting tool, a suction device, and a balloon. For example, FIG. 9(E) shows a balloon 185 being used to apply pressure to bleeding myocardial tissue.

Reference is now made to FIG. 9 and FIG. 11, which are schematic illustrations of apparatus 200 that creates a working space 225 between two layers of tissue, e.g., between the pericardium and myocardium (i.e., within pericardial region 92). Apparatus 200 comprises a flexible longitudinal element 202 shaped to define a lumen thereof, and an expandable element 210 (e.g., an expandable mesh, and/or an inflatable element 206) disposed at a distal portion of flexible longitudinal element 202. Expandable element 210 is shaped to define and at least partly surround working space 225, upon the expandable element being expanded. Apparatus 200 typically further comprises a surgical tool 175 shaped to be passable through the lumen of the flexible longitudinal element and into the working space. In some applications, as shown in FIG. 11, expandable element 210 is shaped to define, upon being expanded, a ring.

Typically, apparatus 200 further comprises an imaging device 24 disposed at least partially at a distal portion of the apparatus. In some applications, imaging device 24 comprises an imaging sensor 24a disposed at the distal portion of the apparatus. (Imaging sensor 24a may be shaped to be passable through the lumen of the flexible longitudinal element.) In other applications (not shown), imaging device 24 comprises a fiber optic array having a distal end that is disposed at the distal portion of the apparatus, and an imaging sensor coupled to a proximal end of the fiber optic array. In some applications, the fiber optic array is shaped to be passable through the lumen of the flexible longitudinal element. Imaging device 24 is typically used to image working space 225 before, during, and/or following the procedure.

As shown in FIG. 11, imaging device 24 may be coupled to flexible longitudinal element 202 and/or to expandable element 210 (e.g., at a distal portion thereof). An advantage of coupling the imaging device to the expandable element (e.g., at the distal portion thereof) is that the working space may be imaged without surgical tool 175 obstructing the imaging device's line of sight.

Apparatus 200 is typically used once access to the pericardial region has been achieved by apparatus 20 or 21, or by any other means. As described hereinabove, apparatus 200 may be advanced over guidewire 70.

FIG. 9 depicts a method for performing a procedure in an area between two layers of tissue, such as in pericardial region 92 (FIG. 5E), which is between the pericardium and myocardium. Working space 225 is created by expanding expandable element 210 in the area such that the expandable element defines and at least partly surrounds the working space. Tool 175 is passed into the working space, and is used to perform the procedure. It is noted that the method depicted in FIG. 9 may be used to create a working space between other layers of tissue, such as between two layers of meninges.

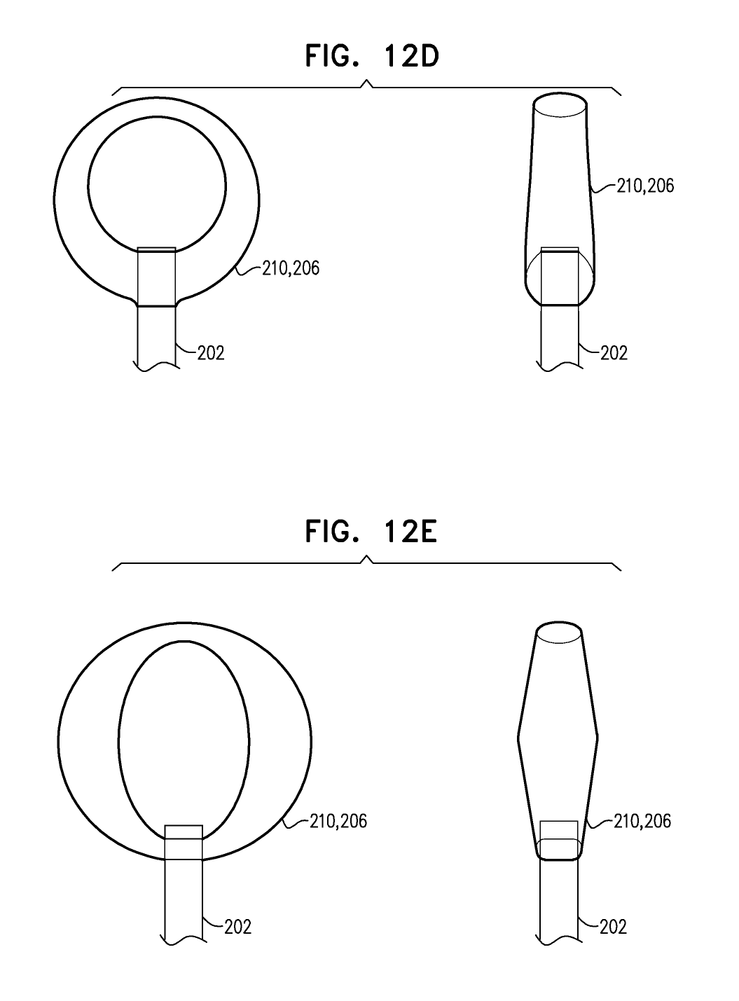

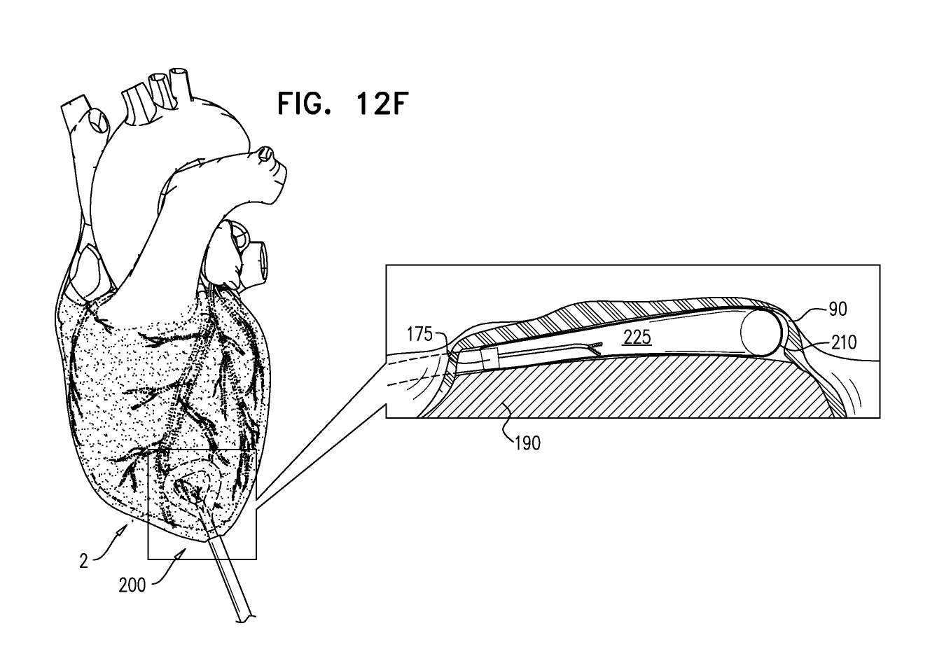

Reference is now made to FIGS. 12A-G, which show respective designs of expandable element 210, in accordance with some applications of the present invention. FIG. 12A shows an application in which expandable element 210 is shaped to define, upon being expanded, a partial ring. In the context of the claims and description of the present application, the term “partial ring” is meant to connote, in a broad sense, any shape that surrounds a space in part, but does not have a closed perimeter. In the particular case shown in FIG. 12A, the partial ring comprises proximal arms 201a and 201b, and distal arras 203a and 203b. The angle between respective proximal and distal arms may be an acute, obtuse, or right angle.

Typically, expandable element is sized and shaped to be containable within a rectangle 208 having (a) a length L between 3 and 8 cm, and (b) a width W between 3 and 8 cm, upon the expandable element being expanded.

FIG. 12B shows an application in which expandable element 210 is shaped to define, upon being expanded, a disk. This disk-shaped expandable element is configured to apply pressure between two layers of tissue, e.g., to inhibit bleeding, upon being expanded. This application differs from the other applications shown in FIGS. 12A-G, in that the disk-shaped expandable element does not define a workspace. Nonetheless, in other respects (e.g., size of the expandable element, use with imaging device 24, etc.), the disk-shaped expandable element is similar to the other types of expandable element.

In some applications, as shown in FIG. 12C, the expandable element is configured to, upon being expanded, have a greater cross-sectional area at a distal portion thereof, relative to a proximal portion thereof. In some applications, the expandable element is configured to, upon being expanded, have a greater cross-sectional area at the proximal portion thereof, relative to the distal portion thereof (FIG. 12D). In some applications, the expandable element is configured to, upon being expanded, have a greater cross-sectional area at a middle portion thereof, relative to (a) the proximal portion thereof, and (b) the distal portion thereof (FIG. 12E). Typically, the portion with the greater cross-sectional area also has a greater height, such that the height of the working space is higher near that portion, relative to other portions of the expandable element. For example, FIG. 12F shows working space 225 having a greater height at the distal portion thereof, relative to the proximal portion thereof, when using the application of expandable element 210 shown in FIG. 12C. In general, having a non-uniform cross-section of the expandable element may facilitate the performance of certain procedures, and/or the use of certain tools.

In some applications, expandable element 210 is an expandable mesh 204 shaped to define a concave shape upon being expanded (FIG. 12G). Tool 175 is inserted through the mesh (e.g., through an opening in the mesh), or underneath the mesh, and the procedure is performed within or the below the concave shape.

In general, the various shapes and designs presented in FIGS. 12A-G may be used in combination with either expandable mesh 204 or inflatable element 206. An advantage of expandable mesh 204 is that the tool may be passed into the working space through the mesh, as shown, for example, in FIG. 12G.

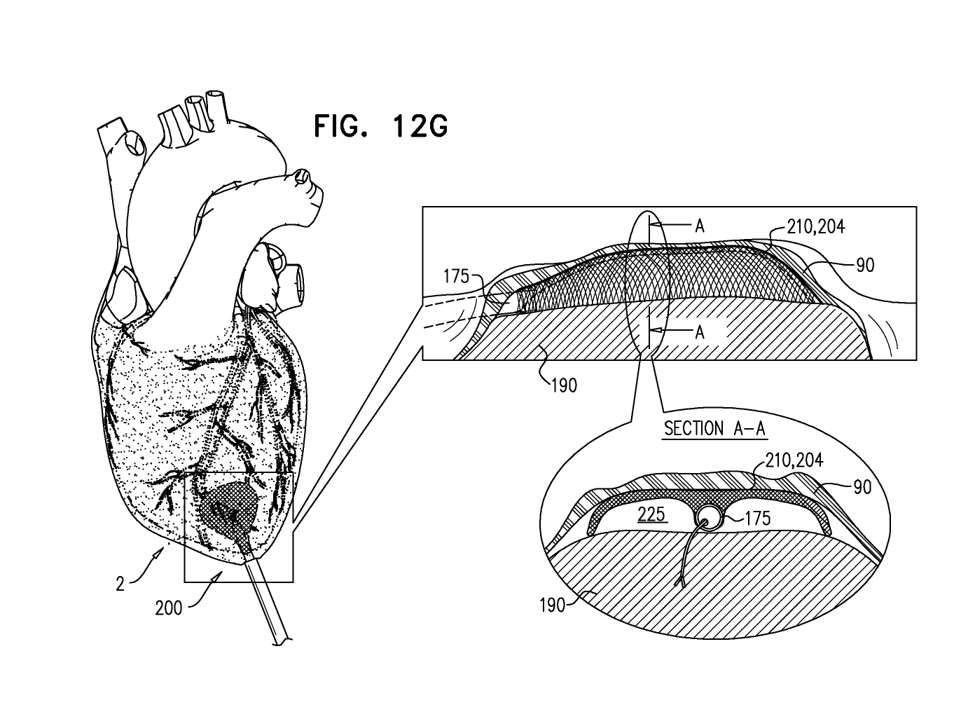

Reference is now made to FIGS. 13A-B, which are schematic illustrations of apparatus 200 being used to reduce flow of blood in a blood vessel 212, in accordance with some applications of the present invention. (The arrows in FIGS. 13A-B indicate the direction of flow of blood.) In some applications, expandable element 210 is used to reduce flow of blood in blood vessel 212, by applying pressure to the blood vessel, e.g., with a proximal portion of the expandable element.

In some applications, flow of blood toward the working space is reduced (FIG. 13A). This reduction in flow may reduce bleeding from the procedure, thus facilitating a safer procedure and faster recovery. For example, in some applications, a left atrial appendage that is at least partially contained within the working space is at least partially removed. In such applications, blood flow toward the working space may be reduced, before, during, and/or following the removal of the appendage. In other applications, flow of blood away from the working space is reduced (FIG. 13B). Such a reduction in flow might be desired, for example, following the application of a suture to blood vessel 212 within working space 225. By applying pressure to the blood vessel with the expandable element, and thus reducing flow of blood away from the working space, a resistance of the suture to pressure may be tested (i.e., the strength of the suture-tissue coupling is tested, and if necessary can be corrected during the same procedure).

In summary, applications of the present invention include performing the following series of steps: (a) creating a working space, by expanding an expandable element in an area between two layers of tissue such that the expandable element defines and at least partly surrounds the working space, (b) passing a tool into the working space, and (c) using the tool to perform a procedure, such as (i) partially removing a left atrial appendage that is at least partially contained within the working space, or (ii) applying a suture to a blood vessel, and testing a resistance of the suture to pressure, by applying pressure to the blood vessel with the expandable element.

Apparatus and techniques described in the following references, each of which is incorporated by reference in the present application, may be combined with apparatus and techniques presented herein:

U.S. patent application Ser. No. 12/780,240, issued as U.S. Pat. No. 8,617,150;

U.S. patent application Ser. No. 14/144,265;

U.S. patent application Ser. No. 13/015,951, published as US 2011/0282203; and

U.S. patent application Ser. No. 13/697,831, published as US 2013/0103028.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
a longitudinal guide member (a) comprising a blunt distal end having an outer surface at least part of which is transparent, and (b) configured to be advanced distally toward a heart of a subject;
a sheath shaped and sized to surround the guide member and shaped to define an at least partially distally-facing suction port at a distal end of the sheath, the apparatus being configured to facilitate drawing a portion of a pericardium of the heart through the suction port and into the sheath, when the suction port is distal to the guide member;
a puncturing element configured to puncture the portion of the pericardium while the portion of the pericardium is in the sheath; and
a puncturing-element-restraining element shaped and positioned with respect to the puncturing element to inhibit passage of a distal tip of the puncturing element out of the distal end of the sheath.

2. The apparatus according to claim 1, wherein at least part of the outer surface the distal end of the guide member is dome-shaped.

3. The apparatus according to claim 1,
wherein the guide member is a guide tube having a proximal end,
wherein the distal end is a distal end of the guide tube, and
wherein the guide tube is shaped to define a guide-tube lumen between the proximal and distal ends of the guide tube.

4. The apparatus according to claim 3, further comprising an imaging sensor disposed within the guide-tube lumen.

5. The apparatus according to claim 3, wherein the guide-tube lumen has a diameter of 4-15 mm.

6. The apparatus according to claim 3, wherein a radius of curvature at a distalmost point of the distal end of the guide tube is less than a radius of the guide tube.

7. The apparatus according to claim 6, wherein the radius of curvature at the distalmost point of the distal end of the guide tube is 30-60% of the radius of the guide tube.

8. The apparatus according to claim 1, wherein the guide member is a shaft shaped to define one or more longitudinal channels therealong.

9. The apparatus according to claim 1, further comprising a puncturing-element tube, wherein the puncturing element is sized and shaped to be passable through the puncturing-element tube and out of a distal end of the puncturing-element tube.

10. The apparatus according to claim 9, wherein the distal end of the puncturing-element tube defines a face that is not perpendicular to a local central longitudinal axis of the puncturing-element tube.

11. The apparatus according to claim 9,
wherein the distal end is shaped to define a puncturing-element-tube hole therein, and
wherein the puncturing-element tube is disposed such that the puncturing-element tube is in contact with a perimeter of the puncturing-element-tube hole.

12. The apparatus according to claim 11, wherein the puncturing-element-tube hole has a diameter of 0.2-2 mm.

13. The apparatus according to claim 11, wherein the puncturing-element-tube hole has a diameter of 2-5 mm.

14. The apparatus according to claim 9, wherein the puncturing-element tube is disposed between the guide member and the sheath.

15. The apparatus according to claim 9, wherein the puncturing-element tube is a puncturing-element-and-suction tube configured to facilitate the drawing of the portion of the pericardium through the suction port of the sheath by application of suction through the puncturing-element-and-suction tube.

16. The apparatus according to claim 1, wherein the puncturing element is disposed between the guide member and the sheath.

17. The apparatus according to claim 1, further comprising a suction tube configured to facilitate the drawing of the portion of the pericardium through the suction port of the sheath by application of suction through the suction tube.

18. The apparatus according to claim 17, wherein a distal end of the suction tube defines a face that is not perpendicular to a local central longitudinal axis of the suction tube.

19. The apparatus according to claim 17, wherein the suction tube is disposed between the guide member and the sheath.

20. The apparatus according to claim 17,
wherein the distal end is shaped to define a suction-tube hole therein, and
wherein the suction tube is disposed such that a distal end of the suction tube is in contact with a perimeter of the suction-tube hole.

21-174. (canceled)
